(19) United States
(12) Patent Application Publication
(10) Pub. No.: US 2024/0117303 A1
(43) Pub. Date: Apr. 11, 2024

(54) METHODS AND COMPOSITIONS FOR GENERATING HUMAN MIDBRAIN DOPAMINERGIC NEURONS FROM NEURAL PROGENITOR CELLS

(71) Applicant: Trailhead Biosystems Inc., Cleveland, OH (US)

(72) Inventor: Nooshin AMINI, Cleveland, OH (US)

(21) Appl. No.: 18/199,415

(22) Filed: May 19, 2023

Related U.S. Application Data
(60) Provisional application No. 63/391,207, filed on Jul. 21, 2022.

(51) Int. Cl. C12N 5/0793

(52) U.S. Cl. CPC C12N 5/0619; C12N 2501/13; C12N 2501/155; C12N 2501/385; C12N 2501/415; C12N 2501/42; C12N 2501/727; C12N 2501/815; C12N 2501/91; C12N 2506/02; C12N 2506/45

(57) ABSTRACT

Methods for generating human midbrain immature neurons and mature dopaminergic neurons from neural progenitor cells are provided. The immature and mature midbrain neurons are obtained from neural progenitors such as committed midbrain neural stem cells (NSCs) and midbrain neural progenitor cells (midbrain NPCs), which themselves are generated from human pluripotent stem cells. The methods for generating midbrain immature and mature neurons use chemically-defined culture media that allow for generation of mature dopaminergic neurons in as little as 23 days. Culture media, isolated cell populations and kits are also provided.

### RELATED APPLICATION

This application claims priority to U.S. Provisional Application No. 63/391,207, filed Jul. 21, 2022, the entire contents of which is hereby incorporated by reference.

### GOVERNMENT LICENSED RIGHTS

This invention was made with government support under Grant Number: W911NF-17-3-0003 awarded by the U.S. ARMY ACC-AGP-RTP. The government has certain rights in the invention.

### BACKGROUND OF THE INVENTION

Parkinson's Disease (PD) is the second most common progressive neurodegenerative disease after Alzheimer's Disease and is characterized by degeneration of midbrain dopamine (mDA) neurons in the substantia nigra pars compacta. Current treatment typically takes a pharmacological approach aimed to increase dopamine bioavailability by administering levodopa (also known as L-dopa), the precursor to dopamine. However, side effects of long-term treatment with levodopa present challenges for its use in later stages of PD. The ability to reconstitute functional dopaminergic neurons in vivo in PD patients was first explored by transplanting human fetal midbrain tissue (reviewed in Lindvall et al. (2004) NeuroRx 1:383-393). Outcomes were variable and the approach raised ethical concerns about the availability and use of fetal tissue, leading to alternative approaches for reconstituting dopaminergic neurons in vivo.

The availability of pluripotent stem cells (PSCs), including embryonic stem (ES) cell lines and induced pluripotent stem cells (iPSCs), opened up the possibility of generating progenitors of mDA neurons in vitro. Developmental studies demonstrated that midbrain dopaminergic neurons originated from the ventral midbrain floor plate (mFP), which can be identified by co-expression of the markers FOXA2 and LMX1A. Early differentiation protocols for deriving midbrain floor plate precursors involved activation of sonic hedgehog (SHH) and canonical WNT signaling in PSCs, as well as dual SMAD inhibition and FGF8 activation, and required 11 days to achieve precursors expressing FOXA2 and LMX1A (Kriks et al. (2011) Nature 480:547-551) or involved activation of SHH, WNT and FGF8, as well as addition of retinoic acid (RA), for a 22-day protocol (Cooper et al. (2010) Mol. Cell. Neurosci. 45:258-266). A similar protocol has been reported in which human iPSCs-derived embryoid bodies were exposed to dual SMAD inhibition for five days, followed by SHH and FGF8 activation, leading to mDA precursors in 16 days (Hartfield et al. (2014) PLoS One 92:e87388).

More recently, additional protocols have been reported for obtaining MB dopaminergic progenitors from human pluripotent stem cells. For example, Nolbrant et al. report a 16 day protocol involving exposure to an N-2 supplement for the first 11 days and a B27 supplement for the last 5 days, as well as SHH and WNT activation and ALK inhibition (Nolbrant et al. (2017) Nature Protocols 12:1962-1979). Precious et al. report a protocol involving MEK inhibition for two days to block FGF signaling, followed by SHH activation alone for three days and SHH and FGF8 activation from day 5 onward, leading to FOXA2+ LMX1A+ progenitors by day 7 (Precious et al. (2020) Front. Neurosci. 14:312). Gartner et al. report a xeno-free, feeder-free chemically-defined protocol that involves incubation in media supplemented with (i) LDN193189 and SB431542 on days 0-5 and LDN193189 alone on days 5-10, (ii) CHIR99021 on days 2-13, and (iii) SHH and purmorphamine on days 1-7 (Gartner et al. (2020) Star Protocols 1:100065).

Human dopaminergic neuron progenitors have also been differentiated from human spermatogonial stem cells (hSSCs) using a protocol involving culture of the hSSCs for four days in olfactory ensheathing cell-conditioned medium (OECCM) supplemented with RA, SB, VPA and forskolin, followed by culture in OECCM supplemented with SHH, FGF8A and TFGrβ3 (Yang et al. (2019) Stem Cell Res. Therap. 10:195).

Methods for expanding midbrain neural progenitor cells also have been described (Fedele et al. (2017) Sci. Reports 7:6036), as have methods of cryopreserving such progenitors (Drummond et al. (2020) Front. Cell. Dev. Biol. 8:578907).

Protocols for differentiating pluripotent stem cells into precursors of midbrain dopaminergic neurons are reviewed in, for example, Arenas et al. (2015) Development 142:1918-1936 and Wang et al. (2020) Cells 9:1489.

Accordingly, while some progress has been, there remains a need for efficient and robust methods and compositions for generating midbrain neural progenitor cells from human pluripotent stem cells and for generating immature and mature dopaminergic neurons from the midbrain neural progenitor cells.

### SUMMARY OF THE INVENTION

This disclosure provides methods of generating immature and mature dopaminergic neurons from neural progenitor cells, such as human committed midbrain (MB) neural stem cells (NSCs) and midbrain neural progenitor cells (NPCs). The neural progenitor cells are obtained from pluripotent stem cells. The culture methods provided herein, using chemically-defined culture media, allows for generation of mature dopaminergic neurons in as little as 23 days of culture starting from human pluripotent stem cells. The culture media comprises small molecule agents that either agonize or antagonize particular signaling pathway activity in the pluripotent stem cells such that differentiation along the midbrain neural lineage is promoted, leading to cellular maturation and expression of midbrain neural progenitor-associated biomarkers, followed by further differentiation and maturation into immature mid-brain neurons by nine days of culture and mature dopaminergic neurons by 23 days of culture. The methods of the disclosure have the advantage that use of small molecule agents in the culture media allows for precise control of the culture components and significantly shortens the differentiation time compared to prior art protocols.

Accordingly, in one aspect, the disclosure pertains to a method of generating human FOXA2+ LMX1A+ MSX1+ PITX3+ DCX+ immature midbrain neurons comprising:
culturing human OTX2+ FOXA2+ LMX1A+ midbrain neural progenitor cells (MB NPCs) in a culture media comprising a WNT pathway agonist, an mTOR pathway antagonist, a retinoic acid receptor (RAR) pathway antagonist, a MEK pathway antagonist, a Notch pathway antagonist and a BMP pathway agonist for three days (day 0-day 3) to obtain human FOXA2+ LMX1A+ MSX1+ PITX3+ DCX+ immature midbrain neurons (MB-immature neurons). This method can further comprise culturing the MB-immature neurons in a culture media comprising a BDNF pathway agonist, a GDNF pathway agonist, a PPAR-a pathway agonist, heparin or heparin mimetic, a Notch pathway antagonist and a dopamine agonist for at least fourteen days to obtain TH+ KCNJ6+ mature dopaminergic neurons.

In another aspect, the disclosure pertains to a two-stage method of generating mature dopaminergic neurons starting from MB NPCs. Accordingly, in one embodiment, the disclosure pertains to a method of generating human TH+ KCNJ6+ mature dopaminergic neurons comprising:
(a) culturing human OTX2+ FOXA2+ LMX1A+ midbrain neural progenitor cells (MB NPCs) in a culture media comprising a WNT pathway agonist, an mTOR pathway antagonist, a retinoic acid receptor (RAR) pathway antagonist, a MEK pathway antagonist, a Notch pathway antagonist and a BMP pathway agonist for three days (day 0-day 3) to obtain human FOXA2+ LMX1A+ MSX1+ PITX3+ DCX+ immature midbrain neurons (MB-immature neurons); and
(b) culturing the MB-immature neurons in a culture media comprising a BDNF pathway agonist, a GDNF pathway agonist, a PPAR-a pathway agonist, heparin or heparin mimetic, a Notch pathway antagonist and a dopamine agonist for at least fourteen days (day 3-day 17) to obtain TH+ KCNJ6+ mature dopaminergic neurons.

In yet another aspect, the disclosure pertains to a four-stage method of generating mature dopaminergic neurons starting from pluripotent stem cells, first generating MB NSCs, followed by MB NPCs, then immature mid-brain neurons and finally mature dopaminergic neurons. Accordingly, in one embodiment, the disclosure pertains to a method of generating human TH+ KCNJ6+ mature dopaminergic neurons comprising:
(a) culturing human pluripotent stem cells in a culture media comprising a WNT pathway agonist, an SHH pathway agonist, a BMP pathway antagonist, an AKT pathway antagonist and a MEK pathway antagonist on days 0-3 to obtain committed midbrain neural stem cells (MB NSCs);
(b) culturing the MB NSCs in a culture media comprising a BMP pathway agonist, an RA pathway agonist, an LXR pathway agonist, an AKT pathway antagonist, an mTOR pathway antagonist and a TGF pathway antagonist on days 4-6 to obtain human OTX2+ FOXA2+ LMX1A+ midbrain neural progenitor cells (MB NPCs);
(c) culturing the MB NPCs in a culture media comprising a WNT pathway agonist, an mTOR pathway antagonist, a retinoic acid receptor (RAR) pathway antagonist, a MEK pathway antagonist, a Notch pathway antagonist and a BMP pathway agonist on days 6-9 to obtain human FOXA2+ LMX1A+ MSX1+ PITX3+ DCX+ immature midbrain neurons (MB-immature neurons); and
(d) culturing the MB-immature neurons in a culture media comprising a BDNF pathway agonist, a GDNF pathway agonist, a PPAR-a pathway agonist, heparin or heparin mimetic, a Notch pathway antagonist and a dopamine agonist on days 9-23 to obtain TH+ KCNJ6+ mature dopaminergic neurons.

In one embodiment, the human pluripotent stem cells are induced pluripotent stem cells (iPSCs). In one embodiment, the human pluripotent stem cells are embryonic stem cells.

In one embodiment, the WNT pathway agonist is CHIR99021. Additional exemplary WNT pathway agonists, along with exemplary concentrations and concentration ranges, are disclosed herein. In one embodiment, the WNT pathway agonist is present in the culture media at a concentration within a range of 0.5-2.0 μM. In one embodiment, the WNT pathway agonist is CHIR99021, which is present in the culture media at a concentration of 1.0-1.1 μM.

In one embodiment, the mTOR pathway antagonist is AZD3147. Additional exemplary mTOR pathway antagonists, along with exemplary concentrations and concentration ranges, are disclosed herein. In one embodiment, the mTOR pathway antagonist is present in the culture media at a concentration within a range of 10-30 nM. In one embodiment, the mTOR pathway antagonist is AZD3147, which is present in the culture media at a concentration of 15 nM.

In one embodiment, the RAR pathway antagonist is AGN193109. Additional exemplary RAR pathway antagonists, along with exemplary concentrations and concentration ranges, are disclosed herein. In one embodiment, the RAR pathway antagonist is present in the culture media at a concentration within a range of 50-250 nM. In one embodiment, the RAR pathway antagonist is AGN193109, which is present in the culture media at a concentration of 100 nM.

In one embodiment, the MEK pathway antagonist is PD0325901. Additional exemplary MEK pathway antagonists, along with exemplary concentrations and concentration ranges, are disclosed herein. In one embodiment, the MEK pathway antagonist is present in the culture media at a concentration within a range of 50-250 nM. In one embodiment, the MEK pathway antagonist is PD0325901, which is present in the culture media at a concentration of 100-110 nM.

In one embodiment, the Notch pathway antagonist is DBZ. Additional exemplary Notch pathway antagonists, along with exemplary concentrations and concentration ranges, are disclosed herein. In one embodiment, the Notch pathway antagonist is present in the culture media at a concentration within a range of 50-250 nM. In one embodiment, the Notch pathway antagonist is DBZ, which is present in the culture media at a concentration of 100 nM.

In one embodiment, the BMP pathway agonist is BMP7. Additional exemplary BMP pathway agonists, along with exemplary concentrations and concentration ranges, are disclosed herein. In one embodiment, the BMP pathway agonist is present in the culture media at a concentration within a range of 5-50 ng/ml. In one embodiment, the BMP pathway agonist is BMP7, which is present in the culture media at a concentration of 10-15 ng/ml.

In one embodiment, the BDNF pathway agonist is BDNF. Additional exemplary BDNF pathway agonists, along with exemplary concentrations and concentration ranges, are disclosed herein. In one embodiment, the BDNF pathway agonist is present in the culture media at a concentration within a range of 5-50 ng/ml. In one embodiment, the BDNF pathway agonist is BDNF, which is present in the culture media at a concentration of 10 ng/ml.

In one embodiment, the GDNF pathway agonist is GDNF. Additional exemplary GDNF pathway agonists, along with exemplary concentrations and concentration ranges, are disclosed herein. In one embodiment, the GDNF pathway agonist is present in the culture media at a concentration within a range of 5-50 ng/ml. In one embodiment, the GDNF pathway agonist is GDNF, which is present in the culture media at a concentration of 10 ng/ml.

In one embodiment, the PPAR-a pathway agonist is GW7647. Additional exemplary PPAR-a pathway agonists, along with exemplary concentrations and concentration ranges, are disclosed herein. In one embodiment, the PPAR-a pathway agonist is present in the culture media at a concentration within a range of 200-300 nM. In one embodiment, the PPAR-a pathway agonist is GW7647, which is present in the culture media at a concentration of 250 nM.

In one embodiment, the heparin or heparin mimetic is heparin. Additional exemplary heparin mimetics, along with exemplary concentrations and concentration ranges, are disclosed herein. In one embodiment, the heparin or heparin mimetic is present in the culture media at a concentration within a range of 2-8 μg/ml. In one embodiment, the culture media comprises heparin, which is present in the culture media at a concentration of 5 μg/ml.

In one embodiment, dopamine agonist is dopamine. Additional exemplary dopamine agonists, along with exemplary concentrations and concentration ranges, are disclosed herein. In one embodiment, the dopamine agonist is present in the culture media at a concentration within a range of 5-15 μM. In one embodiment, the dopamine agonist is dopamine, which is present in the culture media at a concentration of 10 μM.

In another aspect, the disclosure pertains to culture media. In one embodiment, the disclosure pertains to a culture media for obtaining human midbrain immature neurons comprising a WNT pathway agonist, an mTOR pathway antagonist, a retinoic acid receptor (RAR) pathway antagonist, a MEK pathway antagonist, a Notch pathway antagonist and a BMP pathway agonist. In another embodiment, the disclosure pertains to a culture media for obtaining human mature dopaminergic neurons comprising a BDNF pathway agonist, a GDNF pathway agonist, a PPAR-a pathway agonist, heparin or heparin mimetic, a Notch pathway antagonist and a dopamine agonist.

In another aspect, the disclosure pertains to isolated cell cultures. In one embodiment, the disclosure pertains to an isolated cell culture of human midbrain immature neurons, the culture comprising: human FOXA2+ LMX1A+ MSX1+ PITX3+ DCX+ immature midbrain neurons cultured in a culture media comprising a WNT pathway agonist, an mTOR pathway antagonist, a retinoic acid receptor (RAR) pathway antagonist, a MEK pathway antagonist, a Notch pathway antagonist and a BMP pathway agonist. In another embodiment, the disclosure pertains to an isolated cell culture of human mature dopaminergic neurons, the culture comprising: human TH+ KCNJ6+ mature dopaminergic neurons cultured in a culture media comprising a BDNF pathway agonist, a GDNF pathway agonist, a PPAR-a pathway agonist, heparin or heparin mimetic, a Notch pathway antagonist and a dopamine agonist.

Human FOXA2+ LMX1A+ MSX1+ PITX3+ DCX+ immature midbrain neurons generated by any of the methods of the disclosure are also encompassed. Human TH+ KCNJ6+ mature dopaminergic neurons generated by any of the methods of the disclosure are also encompassed.

Other features and advantages of the invention will be apparent from the following detailed description and claims.

### BRIEF DESCRIPTION OF THE DRAWINGS

FIG. 1 shows results from an HD-DoE model of a 13-factor experiment optimized for maximum expression of OTX2. The upper section of the model shows the prediction of expression level of pre-selected 53 genes when optimized for OTX2. The lower section of the model shows the effectors that were tested in this model and their contribution to maximum expression of OTX2. The value column refers to required concentration of each effector to mimic the model.

FIG. 2 shows the results from an HD-DoE model of a 13-factor experiment optimized for maximum expression of FOXA2. Upper and lower sections are as described for FIG. 1. This condition highlights the effector Purmorphamine with factor contribution of 22.2 as an important input for high expression of FOXA2.

FIG. 3 shows the dynamic profile of expression levels of OTX2, LMX1A, DMBX1 and FOXA2 genes relative to the concentration of 13 effectors tested. The positive impact of Purmorphamine, CHIR99021 and LDN193189 on expression of FOXA2 and their factor contribution is shown by the slope of the plots for each effector.

FIG. 4 shows the dynamic profile of expression levels of OTX2, LMX1A, DMBX1 and FOXA2 genes relative to the concentration of 13 effectors tested. The positive impact of MK2206, PD0325901, LDN193189 and CHIR99021 on expression of OTX2 and their factor contribution is shown by the slope of the plots for each effector.

FIG. 5 shows the results from an HD-DoE model of a 12-factor experiment applied on stage 1 neural stem cells to generate a recipe for stage 2 of differentiation. This model is optimized for maximum expression of LMX1A. This setting highlights the role of TTNPB in expression of LMX1A, with factor contribution of 19.5.

FIG. 6 shows the results from an HD-DoE model of a 12-factor experiment applied on stage 1 neural stem cells to generate a recipe for stage 2 of differentiation. This setting highlights the positive role of Purmorphamine and CHIR99021 on expression of FOXA2, with factor contribution of 15.3 and 10.7, respectively.

FIG. 7 shows the dynamic profile of expression levels of LMX1A, FOXA2 and GBX2 genes relative to the concentration of 12 effectors tested. The positive impact of TTNPB, CHIR99021 and GW3965 on expression of LMX1A and of Purmorphamine, MK2206 and GW3965 on the expression of FOXA2 and their factor contribution is shown by the slope of the plots for each effector.

FIG. 8 shows the results from an HD-DoE model of a 12-factor experiment applied on stage 1 neural stem cells to generate a recipe for stage 2 of differentiation. This setting highlights the positive role of BMP7 and MK2206 on expression of FOXA2, with factor contribution of 13.7 and 14.2, respectively.

FIG. 9 shows the results from an HD-DoE model of a 12-factor experiment applied on stage 1 neural stem cells to generate a recipe for stage 2 of differentiation. This setting highlights the positive role of MK2206 and the negative role of FGF8b on expression of LMX1A, with factor contribution of 12 and 16.9, respectively.

FIG. 10 shows the dynamic profile of expression levels of LMX1A, FOXA2 and GBX2 genes relative to the concentration of 12 effectors tested. The positive impact of BMP7 and MK2206 on expression of FOXA2 and of AZD 3147 on the expression of LMX1A and their factor contribution is shown by the slope of the plots for each effector.

FIG. 11A-11B show the dynamic profile of expression levels of OTX2, DMBX1, FOXA2 and LMX1A genes relative to the concentration of 5 validated effectors tested in the recipe of stage 1 of differentiation. FIG. 11A shows the expression levels of genes of interest in the presence of all five finalized effectors. FIG. 11B shows the expression levels of genes of interest in the absence of one of the finalized effectors at a time when others area present.

FIG. 12A-12B show the dynamic profile of expression levels of LMX1A, FOXA2 and GBX2 genes relative to the concentration of 3 validated effectors (TTNPB, A 83-01 and GW3965) tested in the recipe of stage 2 of differentiation. FIG. 12A shows the expression levels of genes of interest in the presence of all three finalized effectors. FIG. 12B shows the expression levels of genes of interest in the absence of one of the finalized effectors at a time when others area present.

FIG. 13A-13B show the dynamic profile of expression levels of LMX1A, FOXA2 and GBX2 genes relative to the concentration of 3 validated effectors (MK2206, AZD 3147 and BMP7) tested in the recipe of stage 2 of differentiation. FIG. 13A shows the expression levels of genes of interest in the presence of all three finalized effectors. FIG. 13B shows the expression levels of genes of interest in the absence of one of the finalized effectors at a time when others area present.

FIG. 14 shows photographs of fluorescence images of MB-derived neural stem cells at the end of stage 1 treatment. Cells were stained with midbrain biomarkers including FOXA2, LMX1A, OTX2, mid-hindbrain boundary biomarker PAX2 and early neuronal biomarker Nestin and hindbrain biomarker GBX2. At this stage cells were positive for all the markers except FOXA2.

FIG. 15 shows photographs of fluorescence images of MB-derived neural stem cells at the end of stage 2 treatment. Cells were stained with midbrain biomarkers including FOXA2, LMX1A, OTX2 and hindbrain biomarker GBX2. At this stage cells were positive for all midbrain biomarkers and very low expression of GBX2 was observed.

FIG. 16 shows a schematic diagram of the two-stage culture protocol for generating midbrain neural progenitor cells from hiPCs in six days, including the stage 1 culture medium for generating midbrain neural stem cells (MB-NSCs) from iPSCs used on day 0 to day 3 and the stage 2 culture medium for generating midbrain neural progenitor cells (MB-NPCs) from MB-NSCs used on day 3 to day 6.

FIG. 17A-17C show RNA-seq data of cells cultured in MB differentiation media after three days (stage 1) and six days (stage 2). FIG. 17A shows a bar plot of differential expression of selected genes at stage 1. At the end of stage 1, expression level of stem cell genes NANOG and POU5F1 were decreased while genes involved in early development of midbrain region and neuronal identity were elevated. FIG. 17B shows a bar plot of differential expression of selected genes at stage 2. At the end of stage 2, expression level of MB progenitor genes including DDC, LMX1B, SOX6 and EN1 increased. FIG. 17C shows a heatmap of gene profile of MB neural progenitors at day 6 compared to gene profile of hiPSCs at day 0.

FIG. 18. shows a schematic diagram of the four-stage culture protocol for generating dopaminergic neurons, including the stage 3 culture medium for generating midbrain immature neurons (MB-immature neurons) from MB-NPCs used on day 6 to day 9 and the stage 4 culture medium for generating dopaminergic neurons from MB-immature neurons used on day 9 to day 23.

FIG. 19 shows the results from an HD-DoE model of a 12-factor experiment optimized for maximum expression of CORIN. The upper section of the model shows the prediction of expression level of pre-selected 53 genes when optimized for CORIN. The lower section of the model shows the effectors that were tested in this model and their contribution to maximum expression of CORIN. The value column refers to required concentration of each effector to mimic the model.

FIG. 20 shows the results from an HD-DoE model of a 12-factor experiment optimized for maximum expression of MSX1. The upper section of the model shows the prediction of expression level of pre-selected 53 genes when optimized for MSX1. The lower section of the model shows the effectors that were tested in this model and their contribution to maximum expression of MSX1. The value column refers to required concentration of each effector to mimic the model. This condition highlights the effector, BMP7 with factor contribution of 11.06 as an important input for high expression of MSX1.

FIG. 21 shows the results from an HD-DoE model of a 12-factor experiment optimized for maximum expression of SOX6. The upper section of the model shows the prediction of expression level of pre-selected 53 genes when optimized for SOX6. The lower section of the model shows the effectors that were tested in this model and their contribution to maximum expression of SOX6. The value column refers to required concentration of each effector to mimic the model. This condition highlights the effector, AGN193109 with factor contribution of 26.1 and PD0325901 with factor contribution of 13.5 as two important inputs for high expression of SOX6.

FIG. 22 shows the results from an HD-DoE model of a 12-factor experiment applied on stage 2 neural progenitors to generate a recipe for stage 3 of differentiation. This model is optimized for maximum expression of KCNJ6. The upper section of the model shows the prediction of expression level of pre-selected 53 genes when optimized for KCNJ6. The lower section of the model shows the effectors that were tested in this model and their contribution to maximum expression of KCNJ6. The value column refers to required concentration of each effector to mimic the model. This setting highlights the role of AGN193109 in expression of KCNJ6 with factor contribution of 17.

FIG. 23 shows the dynamic profile analysis of expression levels of CORIN, MSX1, SOX6 and KCNJ6 relative to concentration of 12 effectors. The positive impact of AGN193109 and CHIR99021 on expression of KCNJ6 and their factor contributions are shown by slope of the plots for each effector.

FIG. 24 shows the results from an HD-DoE model of a 12-factor experiment applied on stage 2 neural progenitors to generate a recipe for stage 3 of differentiation. This setting highlights the positive role of PDBZ and AZD3147 on expression of SOX6 with factor contribution of 21.5 and 20.2, respectively.

FIG. 25 shows the dynamic profile analysis of expression levels of SOX6 and MSX1 relative to concentration of 12 effectors. The positive impact of DBZ, AZD3147 and PD0325901 on expression of SOX6 and negative impact of Activin A and Takinib on expression of MSX1 and their factor contributions are shown by slope of the plots for each effector.

FIG. 26 shows the dynamic profile analysis of expression levels of CORIN, KCNJ6, SOX6 and MSX1 relative to concentration of DBZ and AZD3147. Positive impact of DBZ and AZD3147 on expression of all selected genes and their factor contributions are shown by slope of the plots for each effector.

FIG. 27 shows the results from an HD-DoE model of an 8-factor experiment applied on stage 3 immature neurons to generate a recipe for stage 4 of differentiation. This setting highlights the positive role BDNF and negative role of CHIR99021 on expression of NR4A2 with factor contribution of 17.1 and 20.7 respectively.

FIG. 28 shows the dynamic profile analysis of expression levels of NR4A2 and PITX3 relative to concentration of 8 effectors. The positive impact of BDNF on expression of both genes and negative impact of Rosiglitazone on their expression level, and their factor contributions are shown by slope of the plots for each effector.

FIG. 29A-29B show the dynamic profile of expression levels of CORIN, KCNJ6, MSX1 and SOX6 relative to concentration of four of validated effectors in recipe of stage 3 of differentiation. FIG. 29A shows the expression levels of genes of interest in the presence of finalized effectors. FIG. 29B shows the expression levels of genes of interest in absence of one finalized effector at a time while others are present.

FIG. 30A-30B show the dynamic profile of expression levels of SOX6 and MSX1 relative to concentration of four of validated effectors in recipe of stage 3 differentiation media.

FIG. 30A shows expression levels of genes of interest in the presence of finalized effectors.

FIG. 30B shows expression level of genes of interest in absence of one finalized effector at a time while others are present.

FIG. 31A-31B show the dynamic profile of expression levels of NR4A2 and PITX3 relative to concentration of all six finalized effectors in recipe of stage 4 differentiation media.

FIG. 31A shows expression levels of genes of interest in the presence of all finalized effectors.

FIG. 31B shows expression levels of genes of interest in absence of one finalized effector at a time while others are present.

FIG. 32 shows photographs of the fluorescence images of MB-derived immature neurons at the end of stage 3. Cells are stained with midbrain biomarkers including LMX1A, FOXA2, MSX1, SOX6 and PITX3, and pan neuronal biomarker TUBB3 and immature neuronal marker DCX. At this stage cells were positive for all the markers expected at an immature midbrain neuronal population.

FIG. 33 shows photographs of the fluorescence images of SNc dopaminergic neurons at the end of stage 4. Cells are stained with midbrain biomarkers including TH, KCNJ6 and CALB1 and mature neuronal markers MAP2 and SYN1. At this stage cells were positive for all expected biomarkers and very low expression of CALB1 was observed.

### DETAILED DESCRIPTION OF THE INVENTION

Described herein are methodologies and compositions that allow for the generation of mature dopaminergic neurons from midbrain neural progenitors (themselves generated from human pluripotent stem cells) under chemically-defined culture conditions using a small molecule based approach. The methods of the disclosure generate midbrain neural progenitors in a two stage protocol in which OTX2+ LMX1A+ committed MB neural stem cells (NSCs) are generated in three days, followed by generation of OTX2+ LMX1A+ FOX2A+ MB neural progenitor cells (NPCs) by day six of culture (referred to herein as the stage 1 and 2 recipes). The MB-NPCs then are further differentiated using another two stage protocol (referred to herein as the stage 3 and 4 recipes) to generate immature midbrain neurons by day nine of culture and mature dopaminergic neurons by day 23 of culture. Thus, the disclosure allows for obtention of mature dopaminergic neurons in a significantly shorter time than prior art protocols using chemically-defined culture conditions.

As described in Example 1, a High-Dimensional Design of Experiments (HD-DoE) approach was used to simultaneously test multiple process inputs (e.g., small molecule agonists or antagonists) on output responses, such as gene expression. These experiments allowed for the identification of chemically-defined culture media, comprising agonists and/or antagonists of particular signaling pathways, that is sufficient to generate committed midbrain pluripotent stem cells and midbrain progenitor cells in a very short amount of time. The optimized culture media was further validated by a factor criticality analysis, which examined the effects of eliminating individual agonist or antagonist agents, as described in Example 2. Immunohistochemistry further confirmed the phenotype of the cells generated by the differentiation protocol, as described in Example 3. Furthermore, RNA-seq analysis of cells cultured according to the differentiation protocol also confirmed the expression of MB progenitor genes, as described in Example 4.

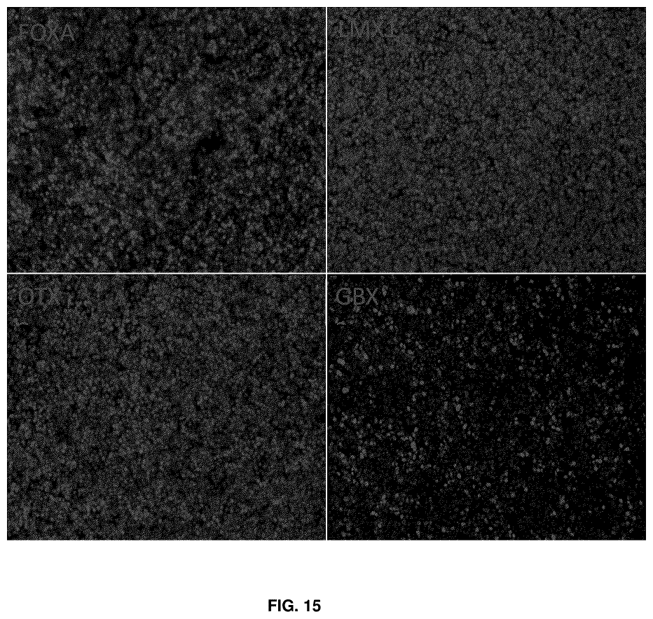

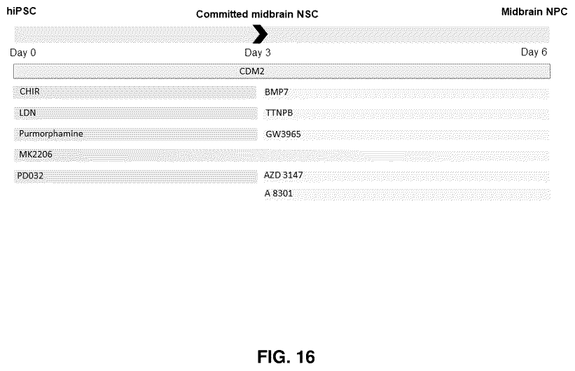

FIG. 16 schematically illustrates an embodiment of the method of the disclosure for generating MB NSCs and MB NPCs.

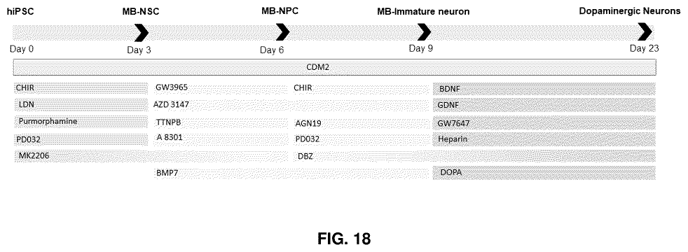

FIG. 18 schematically illustrates an embodiment of the method of the disclosure for generating immature midbrain neurons and mature dopaminergic neurons.

Various aspects of the invention are described in further detail in the following subsections.

### I. Cells

The starting cells used in the cultures of the disclosure are human pluripotent stem cells. As used herein, the term “human pluripotent stem cell” (abbreviated as hPSC) refers to a human stem cell that has the capacity to differentiate into a variety of different cell types. The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, for example, using a nude mouse and teratomas formation assay. Pluripotency can also evidenced by the expression of embryonic stem (ES) cell markers, although the preferred test for pluripotency is the demonstration of the capacity to differentiate into cells of each of the three germ layers.

Human pluripotent stem cells include, for example, induced pluripotent stem cells (iPSC) and human embryonic stem cells, such as ES cell lines. Non-limiting examples of induced pluripotent stem cells (iPSC) include 19-11-1, 19-9-7 or 6-9-9 cells (e.g, as described in Yu, J. et al. (2009) Science 324:797-801). Non-limiting examples of human embryonic stem cell lines include ES03 cells (WiCell Research Institute) and H9 cells (Thomson, J. A. et al. (1998) Science 282:1145-1147). Human pluripotent stem cells (PSCs) express cellular markers that can be used to identify cells as being PSCs. Non-limiting examples of pluripotent stem cell markers include TRA-1-60, TRA-1-81, TRA-2-54, SSEA1, SSEA3, SSEA4, CD9, CD24, OCT3, OCT4, NANOG and/or SOX2. Since the methods of generating committed midbrain neural stem cells and midbrain neural progenitor cells of the disclosure are used to differentiate (maturate) the starting pluripotent stem cell population, in various embodiments the midbrain-committed neural cell populations generated by the methods of the disclosure lack expression of one or more stem cell markers, such as one or more stem cell markers selected from the group consisting of TRA-1-60, TRA-1-81, TRA-2-54, SSEA1, SSEA3, SSEA4, CD9, CD24, OCT3, OCT4, NANOG and/or SOX2

The pluripotent stem cells are subjected to culture conditions, as described herein, that induce cellular differentiation. As used herein, the term “differentiation” refers to the development of a cell from a more primitive stage towards a more mature (i.e. less primitive) cell, typically exhibiting phenotypic features of commitment to a particular cellular lineage.

As used herein, a “neural stem cell” refers to a cell that is more differentiated than a pluripotent stem cell in that it is committed to the neural lineage but still has the capacity to differentiate into different types of cells along the neural lineage.

As used herein a “neural progenitor cell” refers to a cell that is more differentiated than a neural stem cell and that can be further differentiated into a particular type of neural cell.

In embodiments, cells can be identified and characterized based on expression of one or more biomarkers, such as particular biomarkers of neural progenitors or midbrain region-committed neural cells. Non-limiting examples of biomarkers whose expression can be assessed in the characterization of cells of interest include OTX2, which is a mesencephalic marker involved in positioning of midbrain and maintaining the mid-hindbrain boundary (Vernay et al. (2005) J. Neurosci. 25:4856-4867); LMX1A, which is involved in generation and differentiation of midbrain dopaminergic progenitors (Yan et al. (2011) J. Neurosci. 31:12413-12425); FOXA2, which regulates generation of midbrain dopaminergic neurons at early and late stages of development (Ferri et al. (2007) Development 134:2761-2769); PAX2, which is expressed in midbrain and anterior hindbrain (Urbanek et al. (1997) Proc. Natl. Acad. Sci. USA 94:5703-5708); Nestin, which is an early neuronal marker; KI67, which is a proliferation marker; and GBX2, which is a hindbrain marker.

As used herein, expression by a cell of only “low” levels of a biomarker of interest is intended to refer to a level that is at most 20%, and more preferably, less than 20%, less than 15%, less than 10% or less than 5% above background levels (wherein background levels correspond to, for example, the level of expression of a negative control marker that is considered to not be expressed by the cell).

In embodiments, the cells generated by the methods of the disclosure are committed midbrain (MB) neural stem cells (NSCs). As used herein, a “committed midbrain neural stem cell” or “committed MB NSC” refers to a stem cell-derived neural stem cell that expresses the biomarkers OTX2 and LMX1A. In an embodiment, the committed MB NSC does not express, or only expresses low levels of, the biomarker FOXA2. In an embodiment, the committed MB NSC does not express, or only expresses low levels of, the biomarker GBX2. In addition to OTX2 and LMX1A, a committed MB NSC may also express additional biomarkers, including but not limited to PAX2, Nestin and/or KI67.

In embodiments, the cells generated by the methods of the disclosure are midbrain neural progenitor cells, which are more differentiated (more mature) cells than committed MB NSCs. As used herein, a “midbrain neural progenitor cell” or “MB NPC” refers to a stem cell-derived progenitor cell that expresses the biomarkers OTX2, LMX1A and FOXA2. In an embodiment, the MB NPC does not express, or only expresses low levels of, the biomarker GBX2. In addition to OTX2, LMX1A and FOXA2, a MB NPC may also express additional biomarkers, including but not limited to PAX2, Nestin and/or KI67.

The committed MB NSCs and MB NPCs generated by the methods of the disclosure can be further cultured in vitro to generate mature dopaminergic neurons, according to the culture protocols described herein. As used herein, an “immature midbrain neuron” or “MB-immature neuron” refers to a neuronally-derived cell that expresses the biomarkers FOXA2, LMX1A, MSX1, PITX3 and DCX. As used herein, a mature dopaminergic neuron refers to a neuronally-derived cell that expresses the biomarkers TH and KCNJ6, and may also express TUBB3, MAP2, SYN1 and NF.

### II. Culture Media Components

The methods of the disclosure for generating mature dopaminergic neurons, MB-immature neurons, MB NSCs or MB NPCs comprise culturing human pluripotent stem cells in a culture media, often lacking exogenously-added growth factors, and comprising specific agonist and/or antagonists of cellular signaling pathways.

As described herein, a culture media comprising a WNT pathway agonist, an SHH pathway agonist, a BMP pathway antagonist, an AKT pathway antagonist and a MEK pathway antagonist was sufficient to generate OTX2- and LMX1A-expressing MB NSCs in as little as three days (referred to herein as “stage 1” of the differentiation protocol). Further differentiation of the MB NSCs in a culture media comprising a BMP pathway agonist, an RA pathway agonist, an LXR pathway agonist, an AKT pathway antagonist, an mTOR pathway antagonist and a TGF-β pathway antagonist was sufficient to generate OTX2+ FOXA2+ LMX1A+MB NPCs in another three days (referred to herein as “stage 2”), for an overall two-stage six day protocol for generating MB NPCs. Further differentiation of MB NPCs in a culture media comprising a Wnt pathway agonist, mTOR pathway antagonist, a retinoic acid receptor (RAR) antagonist, a MEK pathway antagonist, a Notch pathway antagonist and a BMP pathway agonist for another three days was sufficient to generate FOXA2+, LMX1A+, MSX1+, PITX3+, DCX+ immature MB neurons by day nine of culture (referred to herein as “stage 3”). Finally, further differentiation of immature MB neurons in a culture media comprising a BDNF pathway agonist, a GDNF pathway agonist, a PPAR-a pathway agonist, heparin or heparin mimetic, a Notch pathway antagonist and a dopamine agonist for another 14 days was sufficient to generate TH/KCNJ6+ mature dopaminergic neurons by day 23 of culture (referred to herein as “stage 4”).

As used herein, an “agonist” of a cellular signaling pathway is intended to refer to an agent that stimulates (upregulates) the cellular signaling pathway. Stimulation of the cellular signaling pathway can be initiated extracellularly, for example by use of an agonist that activates a cell surface receptor involved in the signaling pathway (e.g., the agonist can be a receptor ligand). Additionally or alternatively, stimulation of cellular signaling can be initiated intracellularly, for example by use of a small molecule agonist that interacts intracellularly with a component(s) of the signaling pathway.

As used herein, an “antagonist” of a cellular signaling pathway is intended to refer to an agent that inhibits (downregulates) the cellular signaling pathway. Inhibition of the cellular signaling pathway can be initiated extracellularly, for example by use of an antagonist that blocks a cell surface receptor involved in the signaling pathway. Additionally or alternatively, inhibition of cellular signaling can be initiated intracellularly, for example by use of a small molecule antagonist that interacts intracellularly with a component(s) of the signaling pathway.

Agonists and antagonists used in the methods of the disclosure are known in the art and commercially available. They are used in the culture media at a concentration effective to achieve the desired outcome, e.g., generation of midbrain NSCs and/or midbrain NPCs expressing midbrain markers of interest. Non-limiting examples of suitable agonist and antagonists agents, and effective concentration ranges, are described further below.

Agonists of the WNT pathway include agents, molecules, compounds, or substances capable of stimulating (upregulating) the canonical Wnt/r3-catenin signaling pathway, which biologically is activated by binding of a Wnt-protein ligand to a Frizzled family receptor. In one embodiment, a WNT pathway agonist is a glycogen synthase kinase 3 (Gsk3) inhibitor. In one embodiment, the WNT pathway agonist is selected from the group consisting of CHIR99021, CHIR98014, SB 216763, SB 415286, LY2090314, 3F8, A 1070722, AR-A 014418, BIO, BIO-acetoxime, AZD1080, WNT3A, alsterpaullone, indirubin-3-oxime, 1-azakenpaullone, kenpaullone, TC-G 24, TDZD 8, TWS 119, NP 031112, AT 7519, KY 19382, AZD2858, and combinations thereof. In one embodiment, the WNT pathway agonist is present in the culture media at a concentration within a range of 0.3-3.0 μM, 0.5-2.0 μM, 0.75-1.5 μM or 1.0-1.2 μM. In one embodiment, the WNT pathway agonist is CHIR99021. In one embodiment, the WNT pathway agonist is CHIR99021, which is present in the culture media at a concentration within a range of 0.3-3.0 μM, 0.5-2.0 μM, 0.75-1.5 μM or 1.0-1.2 μM. In one embodiment, the WNT pathway agonist is CHIR99021, which is present in the culture media at a concentration of 1.1 μM (e.g., in the stage 1 culture media). In one embodiment, the WNT pathway agonist is CHIR99021, which is present in the culture media at a concentration of 1.0 μM (e.g., in the stage 3 culture media).

Agonists of the SHH (sonic hedgehog) pathway include agents, molecules, compounds, or substances capable of stimulating (activating) signaling through the SHH pathway, which biologically involves binding of SHH to the Patched-1 (PTCH1) receptor and transduction through the Smoothened (SMO) transmembrane protein. In one embodiment, the SHH pathway agonist is selected from the group consisting of Purmorphamine, GSA 10, SAG, and combinations thereof. In one embodiment, the SHH pathway agonist is present in the culture media at a concentration within a range of 100-1000 nM, 200-800 nM, 250-750 nM or 500-600 nM. In one embodiment, the SHH pathway agonist is Purmorphamine. In one embodiment, the SHH pathway agonist is Purmorphamine, which is present in the culture media at a concentration of 100-1000 nM, 200-800 nM, 250-750 nM or 500-600 nM. In one embodiment, the SHH pathway agonist is Purmorphamine, which is present in the culture media at a concentration of 550 nM.

Antagonists of the BMP (bone morphogenetic protein) pathway include agents, molecules, compounds, or substances capable of inhibiting (downregulating) the BMP signaling pathway, which biologically is activated by binding of BMP to a BMP receptor, which are activin receptor-like kinases (ALK) (e.g., type I BMP receptor, including but not limited to ALK2 and ALK3). In one embodiment, the BMP pathway antagonist is selected from the group consisting of LDN193189, DMH1, DMH2, Dorsomorphin, K02288, LDN214117, LDN212854, follistatin, ML347, Noggin, and combinations thereof. In one embodiment, the BMP pathway antagonist is present in the culture media at a concentration within a range of 100-500 nM, 100-400 nM, 150-350 nM or 200-300 nM. In one embodiment, the BMP pathway antagonist is LDN193189. In one embodiment, the BMP pathway antagonist is LDN193189, which is present in the culture media at a concentration within a range of 100-500 nM, 100-400 nM, 150-350 nM or 200-300 nM. In one embodiment, the BMP pathway antagonist is LDN193189, which is present in the culture media at a concentration of 275 nM.

Antagonists of the AKT pathway include agents, molecules, compounds, or substances capable of inhibiting (downregulating) the signaling pathway of one or more of the serine/threonine kinase AKT family members, which include AKT1 (also designated PKB or RacPK), AKT2 (also designated PKB (3 or RacPK-(3) and AKT 3 (also designated PKBy or thyoma viral proto-oncogene 3). In one embodiment, the AKT pathway antagonist is selected from the group consisting of MK2206, GSK690693, Perifosine (KRX-0401), Ipatasertib (GDC-0068), Capivasertib (AZD5363), PF-04691502, AT 7867, Triciribine (NSC154020), ARQ751, Miransertib (ab235550), Borussertib, Cerisertib, and combinations thereof. In one embodiment, the AKT pathway antagonist is present in the culture media at a concentration within a range of 25-300 nM, 50-250 nM, 75-200 nM or 125-150 nM. In one embodiment, the AKT pathway antagonist is MK2206. In one embodiment, the AKT pathway antagonist is MK2206, which is present in the culture media at a concentration within a range of 25-300 nM, 50-250 nM, 75-200 nM or 125-150 nM. In one embodiment, the AKT pathway antagonist is MK2206, which is present in the culture media at a concentration of 138 nM.

In an embodiment, the AKT pathway antagonist present in the culture media in step (a) is the same AKT pathway antagonist present in the culture media in step (b). In an embodiment, the AKT pathway antagonist present in the culture media in step (a) is a different AKT pathway antagonist than the AKT pathway antagonist present in the culture media in step (b). In an embodiment, the AKT pathway antagonist present in the culture media in both step (a) and step (b) is MK2206, e.g., which is present in the culture media in both steps at a concentration within a range of 25-300 nM, 50-250 nM, 75-200 nM or 125-150 nM, such as at 138 nM in both steps.

Antagonists of the MEK pathway include agents, molecules, compounds, or substances capable of inhibiting (downregulating) the signaling pathway of one or more of the components of the MAPK/ERK pathway (also known as the Ras-Raf-MEK-ERK pathway). In one embodiment, the MEK pathway antagonist is selected from the group consisting of PD0325901, Binimetinib (MEK162), Cobimetinib (XL518), Selumetinib, Trametinib (GSK1120212), CI-1040 (PD-184352), Refametinib, ARRY-142886 (AZD-6244), PD98059, U0126, BI-847325, RO 5126766, BIX 02189, Pimasertib, TAK 733, AZD8330, PD318088, SL 327, GDC 0623, RO5126766, Myricetin, and combinations thereof. In one embodiment, the MEK pathway antagonist is present in the culture media at a concentration within a range of 25-300 nM, 50-250 nM, 75-200 nM or 100-120 nM. In one embodiment, the MEK pathway antagonist is PD0325901. In one embodiment, the MEK pathway antagonist is PD0325901, which is present in the culture media at a concentration within a range of 25-300 nM, 50-250 nM, 75-200 nM or 100-120 nM. In one embodiment, the MEK pathway antagonist is PD0325901, which is present in the culture media at a concentration of 110 nM (e.g., in the stage 1 protocol). In one embodiment, the MEK pathway antagonist is PD0325901, which is present in the culture media at a concentration of 100 nM (e.g., in the stage 3 protocol).

Agonists of the RA pathway include agents, molecules, compounds, or substances capable of stimulation of a retinoic acid receptor (RAR) that is activated by both all-trans retinoic acid and 9-cis retinoic acid. There are three RARs: RAR-alpha, RAR-beta and RAR-gamma, which are encoded by the RARA, RARB, RARG genes, respectively. Different retinoic acid analogs have been synthesized that can activate the retinoic acid pathway. Non-limiting examples of such compounds include TTNPB (agonist of RAR-alpha, beta and gamma), AM 580 (RARalpha agonist), CD 1530 (potent and selective RARgamma agonist), CD 2314 (selective RARbeta agonist), Ch 55 (potent RAR agonist), BMS 753 (RARalpha-selective agonist), Tazarotene (receptor-selective retinoid; binds RAR-beta and -gamma), Isotretinoin (endogenous agonist for retinoic acid receptors; inducer of neuronal differentiation), and AC 261066 (RARβ2 agonist). In some embodiments, the RA signaling pathway agonist is selected from the group consisting of: i) a retinoid compound, ii) a retinoid X receptor (RXR) agonist, and iii) a 25 retinoic acid receptor (RARs) agonist. In particular embodiments, the RA pathway agonist is selected from the group consisting of: retinoic acid, Sr11237, adapalene, EC23, 9-cis retinoic acid, 13-cis retinoic acid, 4-oxo retinoic acid, and All-trans Retinoic Acid (ATRA).

Accordingly, in one embodiment, the RA pathway agonist is selected from the group consisting of TTNPB, AM 580, CD 1530, CD 2314, Ch 55, BMS 753, Tazarotene, Isotretinoin, AC 261066, retinoic acid (RA), Sr11237, adapalene, EC23, 9-cis retinoic acid, 13-cis retinoic acid, 4-oxo retinoic acid, and All-trans Retinoic Acid (ATRA), or combinations thereof. In one embodiment, the RA pathway agonist is present in the culture media at a concentration within a range of 5-500 nM, 25-250 nM, 10-100 nM or 25-75 nM. In one embodiment, the RA pathway agonist is TTNPB. In one embodiment, the RA pathway agonist is TTNPB, which is present in the culture media at a concentration within a range of 5-500 nM, 25-250 nM, 10-100 nM or 25-75 nM. In one embodiment, the RA pathway agonist is TTNPB, which is present in the culture media at a concentration of 50 nM.

Antagonists of a retinoic acid receptor pathway include agents, molecules, compounds, or substances capable of inhibiting a retinoic acid receptor (RAR) (i.e., a receptor that is activated by retinoic acid). In one embodiment, the RAR pathway antagonist is selected from the group consisting of AGN193109, BMS 195614, CD 2665, ER 50891, LE 135, LY 2955303, MM11253, and combinations thereof. In one embodiment, the RAR pathway antagonist is present in the culture media at a concentration within a range of 25-300 nM, 50-250 nM, 75-200 nM or 100-120 nM. In one embodiment, the RAR pathway antagonist is AGN193109. In one embodiment, the RAR pathway antagonist is AGN193109, which is present in the culture media at a concentration within a range of 25-300 nM, 50-250 nM, 75-200 nM or 100-120 nM. In one embodiment, the RAR pathway antagonist is AGN193109, which is present in the culture media at a concentration of 100 nM (e.g., in the stage 3 protocol).

Agonists of the LXR (liver X receptor) pathway include agents, molecules, compounds, or substances capable of stimulating (activating) signaling through the LXR pathway, which biologically involves heterodimerization of LXR with the retinoid X receptor (RXR) and activation by oxysterols. In one embodiment, the LXR pathway agonist is selected from the group consisting of GW3965, T0901317, DMHCA, AZ876, and combinations thereof. In one embodiment, the LXR pathway agonist is present in the culture media at a concentration within a range of 100-1000 nM, 200-800 nM, 250-750 nM or 550-650 nM. In one embodiment, the LXR pathway agonist is GW3965. In one embodiment, the LXR pathway agonist is GW3965, which is present in the culture media at a concentration of 100-1000 nM, 200-800 nM, 250-750 nM or 550-650 nM. In one embodiment, the LXR pathway agonist is GW3965, which is present in the culture media at a concentration of 500 nM.

Agonists of the BMP (bone morphogenetic protein) pathway include agents, molecules, compounds, or substances capable of stimulating (upregulating) the BMP signaling pathway, which biologically is activated by binding of BMP to a BMP receptor, which are activin receptor-like kinases (ALK) (e.g., type I BMP receptor, including but not limited to ALK2 and ALK3). In one embodiment, the BMP pathway agonist is selected from the group consisting of BMPs, sb4, ventromorphins (e.g., as described in Genthe et al. (2017) ACS Chem. Biol. 12:2436-2447), and combinations thereof. In one embodiment, the BMP pathway agonist is present in the culture media at a concentration within a range of 1-100 ng/ml, 5-50 ng/ml, 10-25 ng/ml or 12.5-17.5 ng/ml. In one embodiment, the BMP pathway agonist is BMP7. In one embodiment, the BMP pathway agonist is BMP7, which is present in the culture media at a concentration of 1-100 ng/ml, 5-50 ng/ml, 10-25 ng/ml or 12.5-17.5 ng/ml. In one embodiment, the BMP pathway agonist is BMP7, which is present in the culture media in step (b) of the method (i.e., stage 2) at a concentration of 15 ng/ml. In one embodiment, the BMP pathway agonist is BMP7, which is present in the culture media in stage 3 at a concentration of 10 ng/ml.

Antagonists of the TGFβ (transforming growth factor beta) pathway include agents, molecules, compounds, or substances capable of inhibiting (downregulating) signaling through a TGFβ receptor family member, a family of serine/threonine kinase receptors. In one embodiment, the TGFβ pathway antagonist is selected from the group consisting of A 83-01, SB-431542, GW788388, SB525334, TP0427736, RepSox, SD-208, and combinations thereof. In one embodiment, the TGFβ pathway antagonist is present in the culture media at a concentration within a range of 100-500 nM, 200-400 nM, 250-350 nM or 275-325 nM. In one embodiment, the TGFβ pathway antagonist is A 83-01. In one embodiment, the TGF pathway antagonist is A 83-01, which is present in the culture media at a concentration of 100-500 nM, 200-400 nM, 250-350 nM or 275-325 nM. In one embodiment, the TGFβ pathway antagonist is A 83-01, which is present in the culture media in step (b) of the method (i.e., stage 2) at a concentration of 300 nM.

Antagonists of the mTOR (mammalian target of rapamycin) pathway include agents, molecules, compounds, or substances capable of inhibiting (downregulating) signaling through mTOR, a PI3K-related kinase family member which is a core component of the mTORC1 and mTORC2 complexes. In one embodiment, the mTOR pathway antagonist is selected from the group consisting of AZD3147, rapamycin, sirolimus, temsirolimus, everolimus, ridaforolimus, umirolimus, zotarolimus, torin-1, torin-2, vistusertib, AZD8055, dactolisib, PI-103, NU7441, BC-LI-0186, eCF 309, ETP 45658, niclosamide, omipalisib, PF 04691502, PF 05212384, WYE 687, XL 388, STK16-IN-1, PP 242, torkinib, sapanisertib, voxtalisib, and combinations thereof. In one embodiment, the mTOR pathway antagonist is present in the culture media at a concentration within a range of 5-100 nM, 5-50 nM, 10-30 nM or 10-20 nM. In one embodiment, the mTOR pathway antagonist is AZD3147. In one embodiment, the mTOR pathway antagonist is AZD3147, which is present in the culture media at a concentration of 5-100 nM, 5-50 nM, 10-30 nM or 10-20 nM. In one embodiment, the mTOR pathway antagonist is AZD3147, which is present in the culture media in step (b) of the method (i.e., stage 2) at a concentration of 15 nM. In one embodiment, the mTOR pathway antagonist is AZD3147, which is present in the culture media in stage 3 at a concentration of 15 nM.

Antagonists of the Notch pathway include agents, molecules, compounds, or substances capable of inhibiting (downregulating) signaling through a Notch receptor. In one embodiment, the Notch pathway antagonist is selected from the group consisting of DBZ, avagacestat, begacestat, BMS 299897, Compound E, DAPT, JLK6, L-685,458, LY 450139, MRK 560, PF 3084014 hydrobromide, LY 3039478, LY 411575, RO 4929097, and combinations thereof. In one embodiment, the Notch pathway antagonist is present in the culture media at a concentration within a range of 25-300 nM, 50-250 nM, 75-200 nM or 100-120 nM. In one embodiment, the Notch pathway antagonist is DBZ. In one embodiment, the Notch pathway antagonist is DBZ, which is present in the culture media at a concentration within a range of 25-300 nM, 50-250 nM, 75-200 nM or 100-120 nM. In one embodiment, the Notch pathway antagonist is DBZ, which is present in the culture media at a concentration of 100 nM (e.g., in the stage 3 protocol).

Agonists of the brain-derived neurotrophic factor (BDNF) pathway include agents, molecules, compounds, or substances capable of stimulating (upregulating) the BDNF signaling pathway. In one embodiment, the BDNF pathway agonist is selected from the group consisting of BDNF, rotigotine, 7,8-DHF, ketamine, tricyclic dimeric peptide-6 (TDP6), LM22A-4, and combinations thereof. In one embodiment, the BDNF pathway agonist is present in the culture media at a concentration within a range of 1-100 ng/ml, 5-50 ng/ml, 10-25 ng/ml or 12.5-17.5 ng/ml. In one embodiment, the BDNF pathway agonist is BDNF. In one embodiment, the BDNF pathway agonist is BDNF, which is present in the culture media at a concentration of 1-100 ng/ml, 5-50 ng/ml, 10-25 ng/ml or 12.5-17.5 ng/ml. In one embodiment, the BDNF pathway agonist is BDNF, which is present in the culture media in stage 4 of the method at a concentration of 10 ng/ml.

Agonists of the glial cell line-derived neurotrophic factor (GDNF) pathway include agents, molecules, compounds, or substances capable of stimulating (upregulating) the GDNF signaling pathway. In one embodiment, the GDNF pathway agonist is selected from the group consisting of GDNF, BT13, BT44, and combinations thereof. In one embodiment, the GDNF pathway agonist is present in the culture media at a concentration within a range of 1-100 ng/ml, 5-50 ng/ml, 10-25 ng/ml or 12.5-17.5 ng/ml. In one embodiment, the GDNF pathway agonist is GDNF. In one embodiment, the GDNF pathway agonist is GDNF, which is present in the culture media at a concentration of 1-100 ng/ml, 5-50 ng/ml, 10-25 ng/ml or 12.5-17.5 ng/ml. In one embodiment, the GDNF pathway agonist is GDNF, which is present in the culture media in stage 4 of the method at a concentration of 10 ng/ml.

Agonists of the PPAR-a (peroxisome proliferator-activated receptor alpha) pathway include agents, molecules, compounds, or substances capable of stimulating (upregulating) the PPAR-a signaling pathway. In one embodiment, the PPAR-a pathway agonist is selected from the group consisting of GW7647, fenofibrate, fenofibrate-d6, WY 14643, CP 775146, CP 868388 free base, Tesaglitazar, Oleylethanolamide, Oleyolethanolamide-d2, Oleyolethanolamide-d4, PPAR agonist 1, Clofibrate, Clofibrate-d4, Wistin, Indeglitazar, Netoglitazone, GW0742, Bezafibrate, Bezafibrate-d4, Chiglitazar, BMS 687453, Lanifibranor, Saroglitazar, Saroglitazar Magnesium, Saroglitazar-d5, Imiglitazar, AVE-8143, GW 590735, Ertiprotafib, LJ 570, Seladelpar Sodium Salt, Edaglitazone, Muraglitazar, Ragaglitazar, GW 9578, MHY 908, KRP-297, Elafibranor, Aleglitazar, AM3102, Eupatilin, Clofibric acid, Clofibric acid-d4, Naveglitazar, Naveglitazar racemate, and combinations thereof. In one embodiment, the PPAR-a pathway agonist is present in the culture media at a concentration within a range of 50-500 nM, 100-400 nM, 200-300 nM or 225-275 nM. In one embodiment, the PPAR-a pathway agonist is GW7647. In one embodiment, the PPAR-a pathway agonist is GW7647, which is present in the culture media at a concentration of 100-500 nM, 200-400 nM, 250-350 nM or 275-325 nM. In one embodiment, the PPAR-a pathway agonist is GW7647, which is present in the culture media in stage 4 of the method at a concentration of 250 nM.

Heparin is an anticoagulant long known in the art and heparin mimetics are synthetic and semi-synthetic compounds that are highly sulfated, structurally distinct analogues of glycosaminoglycans. In embodiments, the culture media comprises heparin or a heparin mimetic, such as a heparin mimetic selected from the group consisting of heparan sulfate, enoxaparin, small molecular weight heparins, AV5026, M402, and combinations thereof. In one embodiment, heparin or heparin mimetic is present in the culture media at a concentration within a range of 1-10 μg/ml, 2-8 μg/ml, 3-7 μg/ml or 4-6 μg/ml. In one embodiment, the media comprise heparin, which is present in the culture media at a concentration within a range of 1-10 μg/ml, 2-8 μg/ml, 3-7 μg/ml or 4-6 μg/ml. In one embodiment, the media comprise heparin, which is present in the culture media at a concentration of 5 μg/ml.

Dopamine agonists include agents, molecules, compounds, or substances capable of stimulating (upregulating) the dopamine signaling pathway. In embodiments, the dopamine agonist is selected from the group consisting of dopamine, dopamine hydrochloride, (R)-(−)-Apomorphine hydrochloride, Bromocriptine mesylate, Bromocriptine-13C,d3, CNV dopamine, Dehydroergotamine mesylate, Lisuride, Lisuride maleate, Mesulergine hydrochloride, Piribedil dihydrochloride, Piribedil, Quinelorane hydrochloride, (−)-Quinpirole hydrochloride, Ropinirole, Tau-aggregation-IN-1, NMI 8739, U91356, Foscarbidopa, Quinagolide hydrochloride, Dexpramipexole dihydrochloride, PD-168077 maleate, Rotigotine hydrochloride, PF592379, Rotigotine, (Rac)-Rotigotine hydrochloride, (Rac)-Rotigotine-d7 hydrochloride, Ro 10-5824 dihydrochloride, (+)-Dihydrexidine hydrochloride, Talipexole, PF2562, Neuromedin N, A68930, A68930 hydrochloride, A77636 dihydrochloride, PD 119819, PD 128907 hydrochloride, CY 208-243, Dexpramipexole, Dexpramipexole-d3 dihydrochloride, Dexpramipexole-d7 dihydrochloride, SKF 38393 hydrochloride, SKF 38393 hydrobromide, SKF 82958, ABT 670, ABT-724, ABT-724 trihydrochloride, (R)-PF-06256142, Talipexole dihydrochloride, R-Preclamol, Pardoprunox hydrochloride, Pardoprunox, Pergolide mesylate, BP897, BP897 hydrochloride, LY 3154207, Tavapadon, Roxindole, UNC994, Cabergoline, Brexpiprazole, Pergolide-d7 mesylate, Cabergoline-d6, Roxindole hydrochloride, WAY-100635 Maleate, Sibenadet hydrochloride, Dihydrexidine, Dihydrexidine hydrochloride, Bifeprunox, OS-3-106, Brilaroxazine, Pramipexole dihydrochloride hydrate, Pramipexole, Pramipexole dihydrochloride, Cabergoline-d5, Perospirone, Brexpiprazole-d8, (Rac)-Tavapadon, ML417, Brexpiprazole S-oxide, Pramipexole-d7 dihydrochloride, Pramipexole-d5 dihydrochloride, UCSF924, WAY-100635, SKF83959, Pramipexole (N-Propyl-3,3,3-d3) (dihydrochloride), Sarizotan, Brexpiprazole S-oxide D8, SKF 83959 hydrobromide, and combinations thereof. In one embodiment, the dopamine agonist is present in the culture media at a concentration within a range of 5-15 μM, 7.5-12.5 μM, or 9-11 μM. In one embodiment, the dopamine agonist is dopamine. In one embodiment, the dopamine agonist is dopamine, which is present in the culture media at a concentration of 5-15 μM, 7.5-12.5 μM, or 9-11 μM. In one embodiment, the dopamine agonist is dopamine, which is present in the culture media in stage 4 of the method at a concentration of 10 μM.

### III. Culture Conditions

In combination with the chemically-defined and optimized culture media described in subsection II above, the methods of generating mature dopaminergic neurons, immature midbrain neurons, committed MB NSCs and MB NPCs of the disclosure utilize standard culture conditions established in the art for cell culture. For example, cells can be cultured at 37° C. and under 5% O₂ and 5% CO₂ conditions. Cells can be cultured in standard culture vessels or plates, such as 96-well plates. In certain embodiments, the starting pluripotent stem cells are adhered to plates, preferably coated with an extracellular matrix material such as vitronectin. In one embodiment, the stem cells are cultured on a vitronectin coated culture surface (e.g., vitronectin coated 96-well plates).

Pluripotent stem cells can be cultured in commercially-available media prior to differentiation. For example, stem cells can be cultured for at least one day in Essential 8 Flex media (Thermo Fisher #A2858501) prior to the start of the differentiation protocol. In a non-limiting exemplary embodiment, stem cells are passaged onto vitronectin (Thermo Fisher #A14700) coated 96-well plates at 150,000 cells/cm2 density and cultured for one day in Essential 8 Flex media prior to differentiation.

To begin the differentiation protocol, the media the stem cells are being cultured in is changed to a basal differentiation media that has been supplemented with signaling pathway agonists and/or antagonists as described above in subsection II. A basal differentiation media can include, for example, a commercially-available base supplemented with additional standard culture media components needed to maintain cell viability and growth, but lacking serum (the basal differentiation media is a serum-free media) or any other exogenously-added growth factors, such as FGF2, PDGF, IGF or HGF. In a non-limiting exemplary embodiment, a basal differentiation media contains 1×IMDM (Thermo Fisher #12440046), 1×F12 (Thermo Fisher #11765047), poly(vinyl alcohol) (Sigma #p8136) at 1 mg/ml, chemically defined lipid concentrate (Thermo Fisher #11905031) at 1%, 1-thioglycerol (Sigma #M6145) at 450 uM, Insulin (Sigma #11376497001) at 0.7 ug/ml and transferrin (Sigma #10652202001) at 15 ug/ml (also referred to herein as “CDM2” media, as used in the exemplary differentiation protocols shown in FIG. 16 and FIG. 18).

The culture media typically is changed regularly to fresh media. For example, in one embodiment, media is changed every 24 hours.

To generate mature dopaminergic neurons, immature midbrain neurons, committed MB NSCs and MB NPCs, the starting pluripotent stem cells are cultured in the optimized culture media for sufficient time for cellular differentiation and expression of committed MB NSC- or MB NPC-associated markers. As described in the Examples, it has been discovered that culture of pluripotent stem cells in a two-stage method, one optimized for generation of MB NSCs and the other optimized for the generation of MB NPCs, can lead to the production of MB NPCs in as little as six days of culture, with the culture period for the first stage (“stage 1”, leading to MB NSCs) being days 0-3 and the culture period for the second stage (“stage 2”, leading to MB NPCs) being days 4-6. Further culture of the MB NPCs in the stage 3 media for days 6-9 generated immature midbrain neurons (MB-immature neurons), whereas further culture of the MB-immature neurons in the stage 4 media for days 9-23 generated mature dopaminergic neurons.

Accordingly, in the first stage of the method, which generates MB NSCs, also referred to herein as “step (a)” or “stage 1”, pluripotent stem cells are cultured in the stage 1-optimized culture media on days 0-3, or starting on day 0 and continuing through day 3, or for 72 hours (3 days), or for at least 60 hours, or at least 64 hours, or at least 68 hours, or at least 70 hours, or at least 72 hours, or for 60 hours, or for 64 hours, or for 68 hours, or for 70 hours or for 72 hours.

Accordingly, in the second stage of the method, which generates MB NPCs, also referred to herein as “step (b)” or “stage 2”, the MB NSCs generated in step (a) are further cultured in the stage 2-optimized culture media on days 4-6, or starting on day 4 and continuing through day 6, or starting on day 4 and continuing for 72 hours (3 days), or starting on day 4 and continuing for at least 60 hours, or at least 64 hours, or at least 68 hours, or at least 70 hours, or at least 72 hours, or starting on day 4 and continuing for 60 hours, or for 64 hours, or for 68 hours, or for 70 hours or for 72 hours.

Accordingly, in the third stage of the method, which generates immature midbrain neurons, also referred to herein as “step (c)” or “stage 3”, the MB NPCs generated in step (b) are further cultured in the stage 3-optimized culture media on days 6-9, or starting on day 6 and continuing through day 9, or starting on day 6 and continuing for 72 hours (3 days), or starting on day 6 and continuing for at least 60 hours, or at least 64 hours, or at least 68 hours, or at least 70 hours, or at least 72 hours, or starting on day 6 and continuing for 60 hours, or for 64 hours, or for 68 hours, or for 70 hours or for 72 hours.

Accordingly, in the fourth stage of the method, which generates mature dopaminergic neurons, also referred to herein as “step (d)” or “stage 4”, the immature midbrain neurons generated in step (c) are further cultured in the stage 4-optimized culture media on days 9-23, or starting on day 9 and continuing through day 23, or starting on day 9 and continuing in culture for sufficient time to generate TH+ KCNJ6+ mature dopaminergic neurons (e.g., 14 days, or two weeks, of culture in the stage 4 media).

### IV. Uses

The methods and compositions of the disclosure for generating mature dopaminergic neurons, immature midbrain neurons, committed MB NSCs and MB NPCs allow for efficient and robust availability of these cell populations for a variety of uses. For example, the methods and compositions can be used in the study of midbrain neural progenitor development and biology, including differentiation into dopaminergic neurons, to assist in the understanding and potential treatment of neuronal diseases and disorders such as Parkinson's disease. For example, the mature dopaminergic neurons, immature midbrain neurons, committed MB NSCs and/or MB NPCs generated using the methods of the disclosure can be further purified according to methods established in the art using agents that bind to surface markers expressed on the cells. Accordingly, in one embodiment, the disclosure provides a method of isolating mature dopaminergic neurons or immature midbrain neurons, the method comprising: contacting mature dopaminergic neurons or immature midbrain neurons generated by a method of the disclosure with at least one binding agent that binds to a cell surface marker expressed by the mature dopaminergic neurons or immature midbrain neurons; and isolating cells that bind to the binding agent to thereby isolate the mature dopaminergic neurons or immature midbrain neurons.

In one embodiment, the binding agent is an antibody, e.g., a monoclonal antibody (mAb) that binds to the cell surface marker. Cells that bind the antibody can be isolated by methods known in the art, including but not limited to fluorescent activated cell-sorting (FACS) and magnetic activated cell sorting (MACS).

Progenitors of the midbrain dopaminergic neural lineage also are contemplated for use in the treatment of neural diseases and disorders that would benefit from enhancement of dopaminergic neuronal function, through delivery of the cells to a subject having the disease or disorder, including but not limited to Parkinson's Disease.

The cells of the disclosure also are useful in the screening of potential drugs or for the development of novel cell therapies for the treatment of diseases or disorders involving dysfunction of dopaminergic neurons.

### V. Compositions

In other aspects, the disclosure provides compositions related to the methods of generating mature dopaminergic neurons, immature midbrain neurons, committed MB NSCs and MB NPCs, including culture media and cell cultures, as well as isolated progenitor cells and cell populations.

In one aspect, the disclosure provides a culture media for obtaining human committed midbrain neural stem cells comprising a WNT pathway agonist, an SHH pathway agonist, a BMP pathway antagonist, an AKT pathway antagonist and a MEK pathway antagonist. In an embodiment, the culture media lacks exogenously-added growth factors.

In another aspect, the disclosure provides a culture media for obtaining human midbrain neural progenitor cells comprising a BMP pathway agonist, an RA pathway agonist, an LXR pathway agonist, an AKT pathway antagonist, an mTOR pathway antagonist and a TGF-β pathway antagonist. In an embodiment, the culture media lacks exogenously-added growth factors.

In another aspect, the disclosure provides a culture media for obtaining human midbrain immature neurons comprising a WNT pathway agonist, an mTOR pathway antagonist, a retinoic acid receptor (RAR) pathway antagonist, a MEK pathway antagonist, a Notch pathway antagonist and a BMP pathway agonist. In an embodiment, the culture media lacks exogenously-added growth factors.

In another aspect, the disclosure provides a culture media for obtaining human mature dopaminergic neurons comprising a BDNF pathway agonist, a GDNF pathway agonist, a PPAR-a pathway agonist, heparin or heparin mimetic, a Notch pathway antagonist and a dopamine agonist. In an embodiment, the culture media lacks exogenously-added growth factors.

In another aspect, the disclosure provides an isolated cell culture of human committed midbrain neural stem cells, the culture comprising: human OTX2+ LMX1A+ committed midbrain neural stem cells cultured in a culture media comprising a WNT pathway agonist, an SHH pathway agonist, a BMP pathway antagonist, an AKT pathway antagonist and a MEK pathway antagonist and lacking exogenously-added growth factors.

In another aspect, the disclosure provides an isolated cell culture of human midbrain neural progenitor cells, the culture comprising: human OTX2+ FOXA2+ LMX1A+ midbrain neural progenitor cells cultured in a culture media comprising a BMP pathway agonist, an RA pathway agonist, an LXR pathway agonist, an AKT pathway antagonist, an mTOR pathway antagonist and a TGF-β pathway antagonist, and lacking exogenously-added growth factors.

In another aspect, the disclosure provides an isolated cell culture of human midbrain immature neurons, the culture comprising: human FOXA2+ LMX1A+ MSX1+ PITX3+ DCX+ immature midbrain neurons cultured in a culture media comprising a WNT pathway agonist, an mTOR pathway antagonist, a retinoic acid receptor (RAR) pathway antagonist, a MEK pathway antagonist, a Notch pathway antagonist and a BMP pathway agonist.

In another aspect, the disclosure provides an isolated cell culture of human mature dopaminergic neurons, the culture comprising: human TH+ KCNJ6+ mature dopaminergic neurons cultured in a culture media comprising a BDNF pathway agonist, a GDNF pathway agonist, a PPAR-a pathway agonist, heparin or heparin mimetic, a Notch pathway antagonist and a dopamine agonist.

In another aspect, the disclosure provides a human OTX2+ FOXA2+ LMX1A+ midbrain neural progenitor cells generated by a method of the disclosure. In an embodiment, the disclosure pertains to a composition comprising a human midbrain neural progenitor cell (NPC), wherein the human midbrain NPC expresses OTX2, FOXA2 and LMX1A and lacks expression of, or has only low levels of expression of, GBX2. In an embodiment, the disclosure pertains to an isolated cell population of human midbrain neural progenitor cells (NPCs) comprising at least 1×10⁶ OTX2+ FOXA2+ LMX1A+ human midbrain NPCs, wherein the cell population lacks GBX2-expressing neural stem cells. In an embodiment of the isolated cell population, the human midbrain NPCs are bound with at least one antibody that binds at least one marker expressed by the human midbrain NPCs.

In other aspect, the disclosure provides human FOXA2+ LMX1A+ MSX1+ PITX3+ DCX+ immature midbrain neurons generated by a method as described herein. In another aspect, the disclosure provides human TH+ KCNJ6+ mature dopaminergic neurons generated by a method as described herein.

The present invention is further illustrated by the following examples, which should not be construed as further limiting. The contents of figures and all references, patents and published patent applications cited throughout this application are expressly incorporated herein by reference.

### EXAMPLES

### Example 1: Culture Protocol Development for the Generation of Stem Cell-Derived Midbrain Neural Progenitors Expressing FOXA2 and LMX1A

In this example, a two-stage culture protocol for generation of midbrain-derived neural progenitors was developed that can guide human pluripotent stem cells to progenitors expressing FOXA2 and LMX1A after 6 days in culture. These cells can be further differentiated to mature dopaminergic neurons.

This example utilizes a method of High-Dimensional Design of Experiments (HD-DoE), as previously described in Bukys et al. (2020) Iscience 23:101346. The method employs computerized design geometries to simultaneously test multiple process inputs and offers mathematical modeling of a deep effector/response space. The method allows for finding combinatorial signaling inputs that control a complex process, such as during cell differentiation. It allows testing of multiple plausible critical process parameters, as such parameters impact output responses, such as gene expression. Because gene expression provides hallmark features of the phenotype of, for example, a human cell, the method can be applied to identify, and understand, which signaling pathways control cell fate. In the current example, the HD-DOE method was applied with the intent to find conditions for induction of midbrain neural progenitor-expressed genes, directly from the pluripotent stem cell state.

To develop the recipe for each stage, the impact of agonists and antagonists of multiple signaling pathways (referred to herein as “effectors”) on the expression of two sets of 53 pre-selected genes after a 3-day treatment was tested and modeled. These effectors are small molecules or proteins that are commonly used during stepwise differentiation of stem cells to specific fates. The choice of the effectors to test was based on current literature on neural induction in the midbrain region of the developing brain and differentiation of stem cells to neural progenitors.

To test the effectors, experiments with at least 8 factors were designed that can assess the response of cells to 48 or more different combinations of effectors in a range of concentrations. To analyze the models, we focused on expression of genes expressed in the midbrain region, including OTX2, DMBX1, FOXA2, LMX1A, and on the absence of GBX2, which is a hindbrain marker. The impact of each effector on gene expression level is defined by a parameter called factor contribution that is calculated for each effector during the modeling.

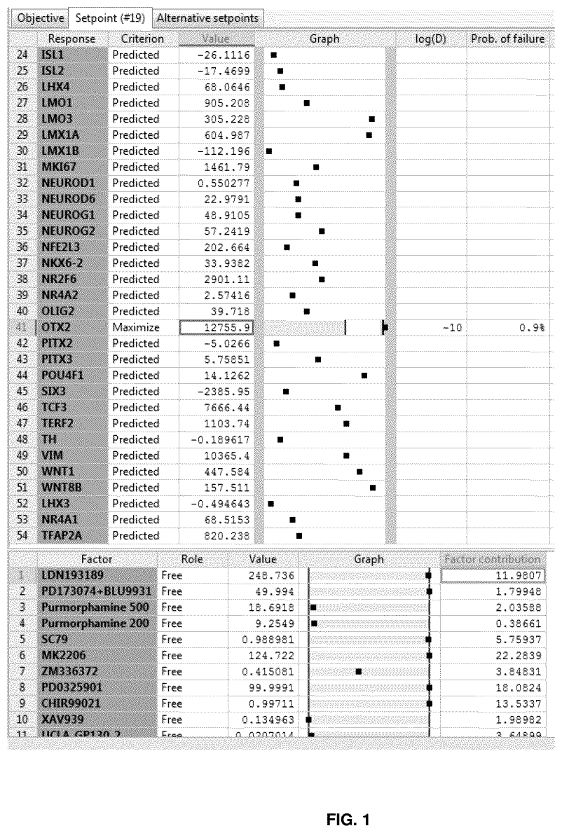

To identify the recipe of stage 1 of differentiation, cells were treated with various effectors for 3 days and the gene expression of cells was modeled. One model specifically, showed promising results on upregulation of DMBX1, LMX1A and OTX2 and downregulation of GBX2, when optimized for maximum expression of OTX2 at 12760.1. This model consisted of 13 factors including LDN193189, PD173074, BLU9931, Purmorphamine, SC79, MK2206, ZM336372, PD0325901, CHIR99021, XAV939, UCLA-gp130, Tofacitinib and GO 6983. Four of the effectors, MK2206 which is an antagonist of AKT signaling pathway, PD0325901 which is an antagonist of MEK signaling pathway, CHIR99021 which is an agonist of WNT signaling pathway and LDN193189 which is an antagonist of BMP signaling pathway had significant positive impact on expression of genes of interest with 22.3, 18.1, 13.5 and 11.9 factor contributions, respectively (FIG. 1).

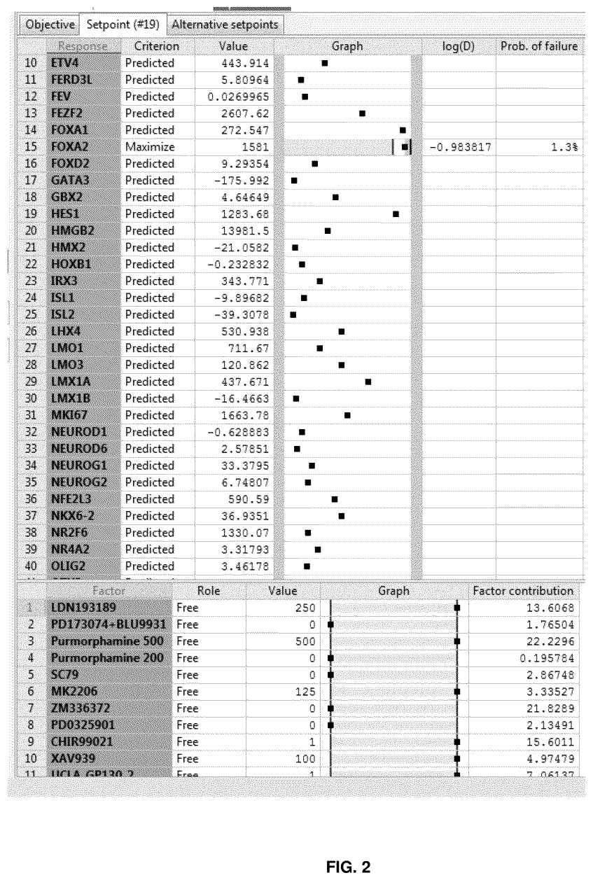

Since FOXA2 was not upregulated with optimization of OTX2, we next optimized the model for maximum expression of FOXA2 at 1581. Three effectors with significant positive effect on expression of FOXA2 were identified including LDN193189, CHIR99021 and Purmorphamine with 13.6, 15.6 and 22.2 factor contribution, respectively (FIG. 2). LDN193189 and CHIR99021 are common in both optimization settings and the rest of the factors except Purmorphamine, have a factor contribution less than 10. Therefore, we focused on impact of addition of Purmorphamine to the original 4 effectors.

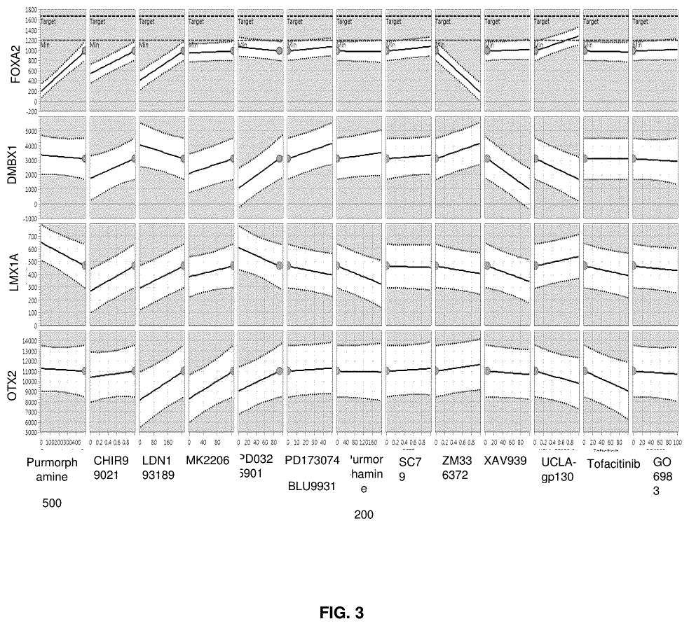

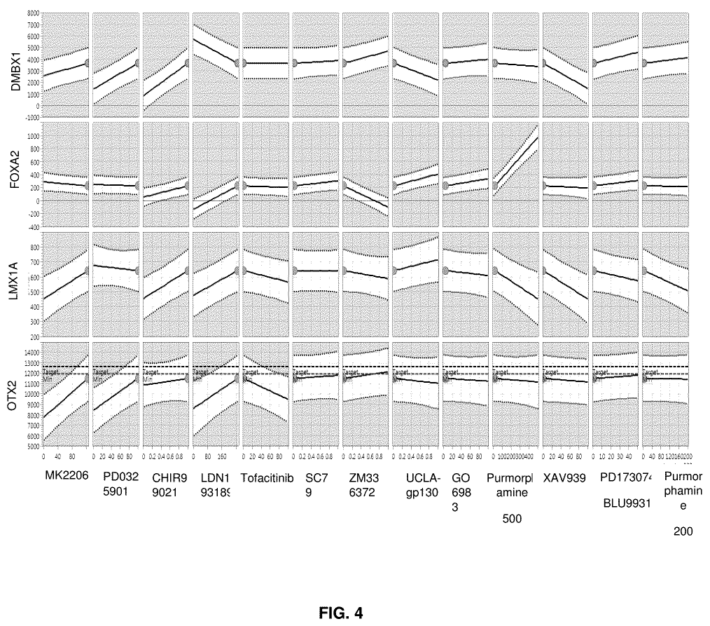

This assessment was done through dynamic profile analysis of the model with focus on expression of OTX2, DMBX1, LMX1A and FOXA2 (FIG. 3). Since Purmorphamine did not have a positive impact on expression of OTX2, DMBX1 and LMX1A in previous setting, we expected a drop in the expression level of the genes, however, it was observed that their expression level stayed in the same vicinity as the previous condition with 3000, 450 and 11000 for DMBX1, LMX1A and OTX2, respectively (FIG. 4).

The effectors validated for Stage 1 of the Protocol (generating midbrain committed neural stem cells) are summarized below in Table 1:

TABLE 1
Validated Effectors in Stage 1 of Protocol

| Effectors | Role | Concentration |
|---|---|---|
| LDN193189 | BMP antagonist | 275 nM |
| PD0325901 | MEK antagonist | 110 nM |
| CHIR99021 | WNT agonist | 1.1 uM |
| MK2206 | AKT antagonist | 138 nM |
| Purmorphamine | SHH agonist | 550 nM |

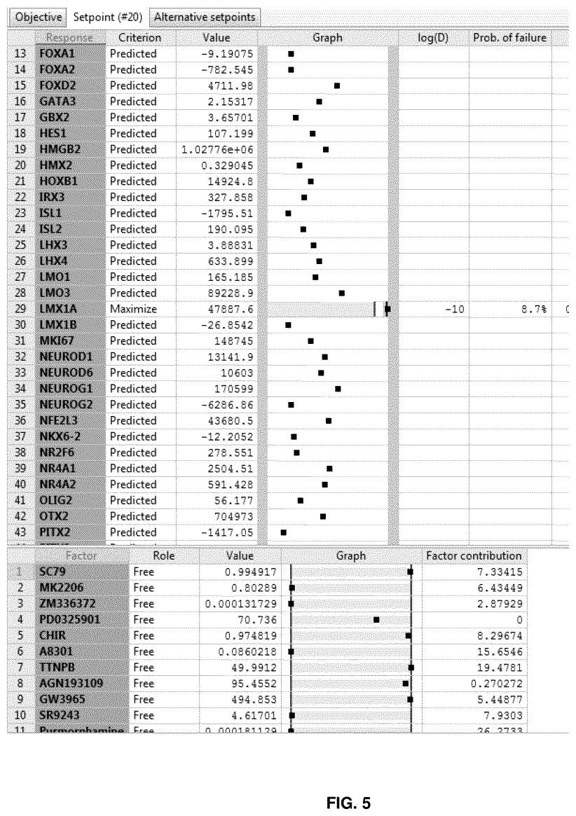

To further guide the differentiation of midbrain-committed neural stem cells to neural progenitor cells at stage 2, we performed an additional HD-DoE experiment. We thereby obtained additional gene regulatory models that were used for preparation of differentiation protocol. The basis of this was a 12-factor HD-DoE experiment with focus on initiation of differentiation of cells toward midbrain neural progenitor cells for an additional 3 days after termination of stage 1 treatment. Here, we focused on expression of LMX1A and FOXA2 in neural progenitor cells with low to zero expression of GBX2. LMX1A had significant high expression level in the model with value of 47888. Therefore, we used optimization setting for this gene to identify the positive factors. The factors in this experiment included SC79, MK2206, ZM336372, PD0325901, CHIR99021, A 83-01, TTNPB, AGN193109, GW3965, SR9243, Purmorphamine and GSI-XX. When optimized for LMX1A, one factor, TTNPB, which is a small molecule agonist of RA signaling pathway, had significant positive impact with factor contribution of 19.5. CHIR99021, SC79 and GW3965 also had positive impacts but their factor contribution was less than 10 (8.3, 7.3 and 5.4 respectively) and AGN193109 had <1 positive factor contribution (FIG. 5).

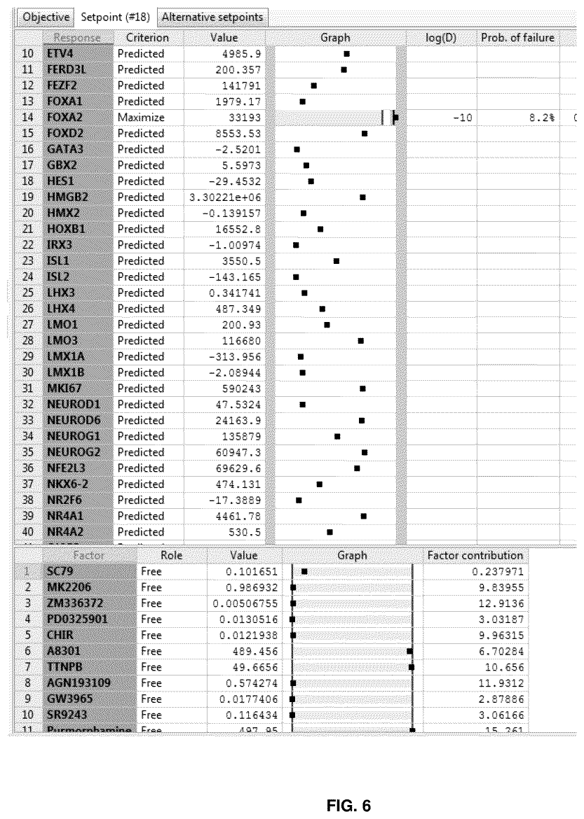

When the same experiment was optimized for maximum expression of FOXA2 at 33193, three effectors were identified with significant positive impact on expression of FOXA2 which include TTNPB, A 83-01 and Purmorphamine with 10.7, 6.7 and 15.3 factor contribution (FIG. 6).

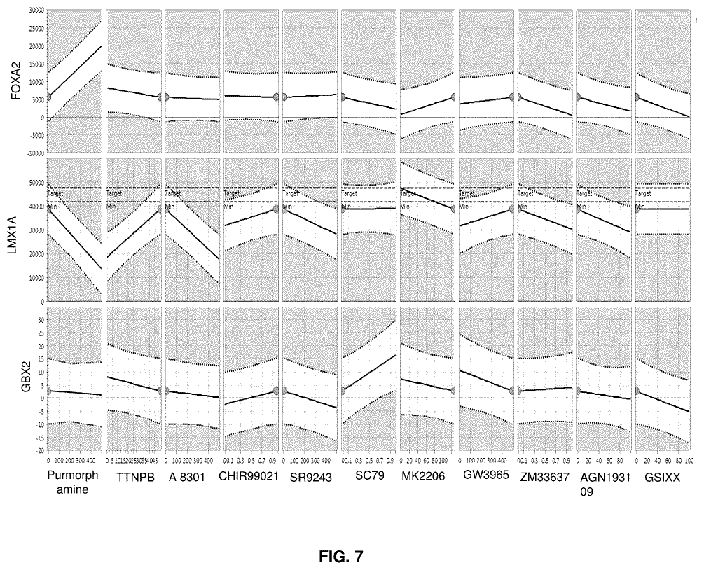

Similar to the experimental model of stage 1, the analysis again showed that Purmorphamine had a positive effect on FOXA2 expression levels and a negative effect on LMX1A expression levels, with factor contribution of 26.2. The model also revealed the same trend for A 83-01 and CHIR99021, with positive impacts only on FOXA2 and LMX1A, respectively. Therefore, we used dynamic profile analysis to adjust the recipe for optimized expression of both LMX1A and FOXA2 genes and minimum expression of GBX2 (FIG. 7). It was observed that even without addition of Purmorphamine, expression level of FOXA2 was at 5000, therefore inclusion of this effector is not essential, however it can improve expression of FOXA2. Based on dynamic profile plots, it was also concluded that even though CHIR99021 has positive effect on LMX1A, it also increases the expression level of GBX2 and reduces the relative expression of FOXA2 and therefore this factor was eliminated in the final recipe. A 83-01 was another factor with opposite effect on FOXA2 and LMX1A that was included in the final recipe. The dynamic profile analysis showed that addition of A 83-01 at a moderate concentration (300 nM) can improve expression of FOXA2 and help reducing the level of GBX2 to almost 0.

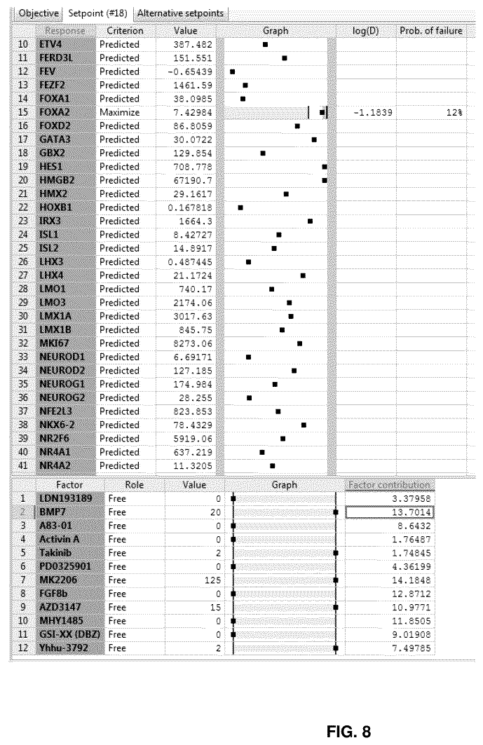

To test additional factors like FGF8 that is routinely used in midbrain differentiation protocols, we ran another 12-factor experiment consisting of LDN193189, BMP7, A 83-01, Activin A, Takinib, PD0325901, MK2206, FGF8b, AZD3147, MHY1485, GSI-XX and Yhhu 3792. Similar to the previous experiment, hiPSCs were treated with stage 1 media for 3 days and then treated with 96 conditions of combinations of the factors for an additional 3 days. When this model was optimized for maximum expression of FOXA2, BMP7 with factor contribution of 13.7 and MK2206 with 14.2 had the most impact on its expression followed by AZD 3147 with factor contribution of 10.9. Yhhu 3792 and takinib also had positive impacts but factor contributions were less than 10. Surprisingly, FGF8 had negative impact with factor contribution of 12.8 (FIG. 8).

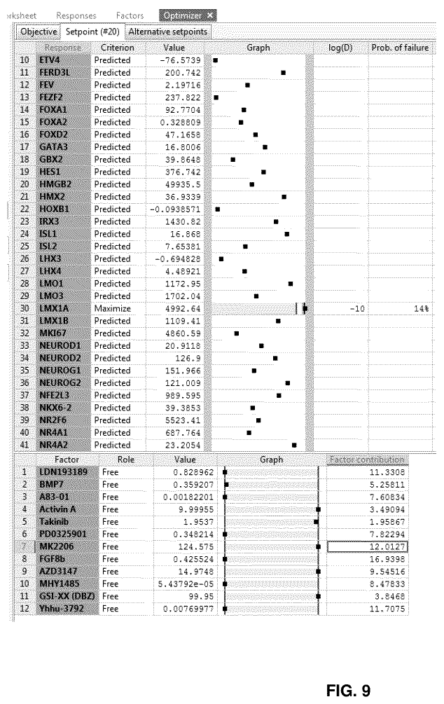

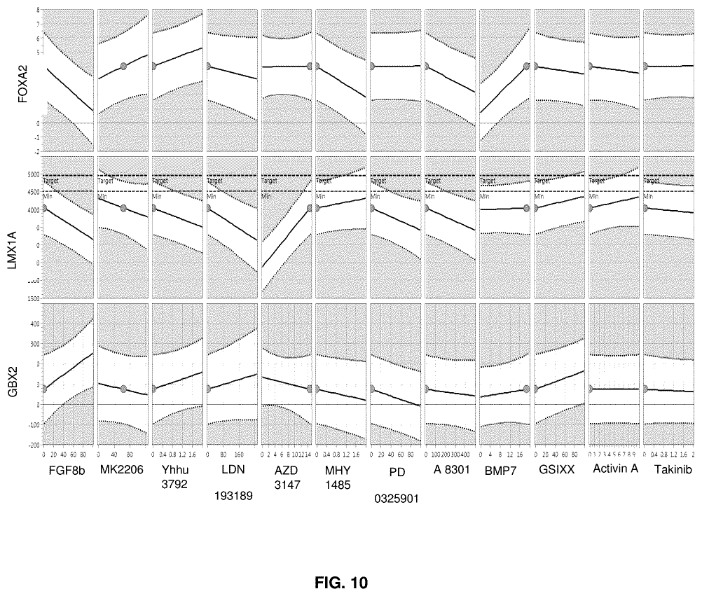

This model was also optimized for maximum expression of LMX1A, and MK2206 with factor contribution of 12 had the highest positive impact. AZD 3147, GSI-XX, Activin A and Takinib also had positive impact on its expression, but the factor contributions were less than 10 (FIG. 9). The model showed that Yhhu 3792 had a negative impact on expression of LMX1A, with factor contribution of 11.7, which is the opposite of FOXA2 condition. Another difference was BMP7, which has a negative impact on LMX1A. However, the factor contribution is less than 10. Therefore, to evaluate the effect of interactions between the factors and the optimal condition for the expression of both FOXA2 and LMX1A, we used dynamic profile analysis (FIG. 10).

Using dynamic profile analysis, we eliminated GSI-XX, Activin A and Takinib, since they did not make a meaningful positive change in the level of both FOXA2 and LMX1A expression. Yhhu 3792 was also eliminated since it had a considerable negative effect on LMX1A and positive effect on GBX2. BMP7 and AZD 3147 had significant positive impact on FOXA2 and LMX1A, respectively, while they did not reduce the expression of the other gene, therefore they were included in the final recipe. It was also shown that even though MK2206 decreases the level of LMX1A, it has desirable impact on FOXA2 and GBX2, therefore it was included in the final recipe at a moderate level.

The effectors validated for Stage 2 of the Protocol (generating midbrain-derived neural progenitor cells) are summarized below in Table 2:

TABLE 2
Validated Effectors in Stage 2 of Protocol

| Effectors | Role | Concentration |
|---|---|---|
| TTNPB | RA agonist | 50 nM |
| BMP7 | BMP pathway | 15 ng/ml |
| GW3965 | LXR agonist | 500 nM |
| A 83-01 | TGF-β antagonist | 300 nM |
| AZD 3147 | mTOR antagonist | 15 nM |
| MK2206 | AKT antagonist | 50 nM |

Considering both models, conditions that maximize differentiation of cells to the midbrain region with neural progenitor cell identity as such relate to robust and elevated expression of OTX2, FOXA2 and LMX1A included the following effector inputs: TTNPB, BMP7, A 83-01, GW3965, AZD 3147 and MK2206.

The criticality of each individual validated effector for the stage 1 and stage 2 protocols was further evaluated as described in Example 2.

### Example 2: Factor Criticality Analysis of Stem Cell Derived Midbrain Neural Progenitor-Inducing Culture Conditions

To assess the impact of elimination of each validated factor, we used dynamic profile analysis and compared the expression level of genes of interest in absence of each finalized factor while others are present. Since expression level of genes of interest reveal whether the desired outcome is reachable, this factor criticality analysis revealed the extent of importance of each input effector.

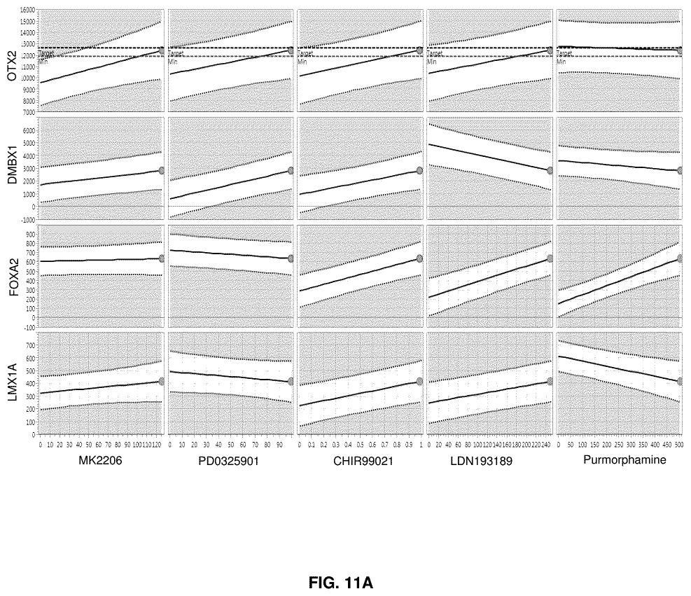

In the stage 1 recipe, each of the five finalized factors were removed while the other four factors were present and the expression levels of OTX2, DMBX1, FOXA2 and LMX1A was assessed compared to the levels in the presence of all five factors (FIG. 11A-B). When MK2206 was removed, values of OTX2 and DMBX1 decreased from 12000 and 3000 to 9500 and 1500, respectively, while FOXA2 and LMX1A stayed the same. Absence of PD0325901 resulted in reduced expression of DMBX1 and it reached 900 while expression of FOXA2 and LMX1A increased from 600 and 300 to 700 and 500. Expression level of DMBX1 increased in absence of LDN193189, however values of OTX2, FOXA2 and LMX1A decreased. Values of all genes of interest was reduced after removing CHIR99021 which further proved its importance in stage 1 recipe and, as expected, absence of Purmorphamine lead to reduction of FOXA2 while it was beneficial for other genes.

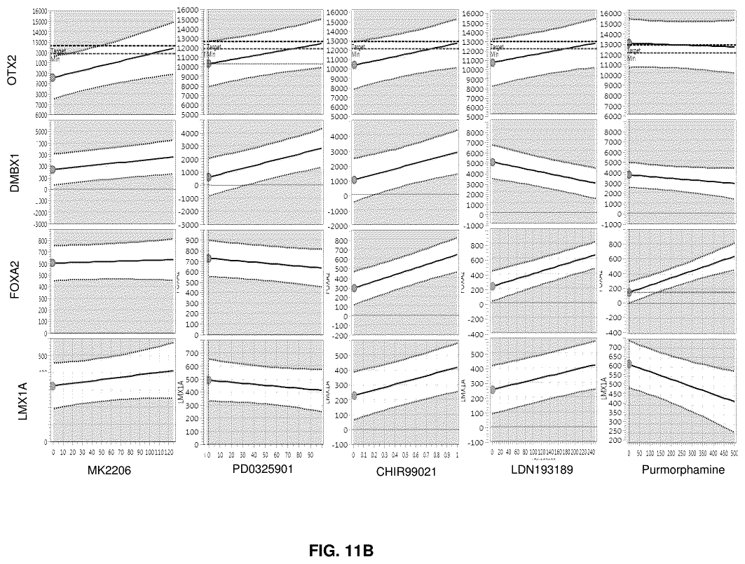

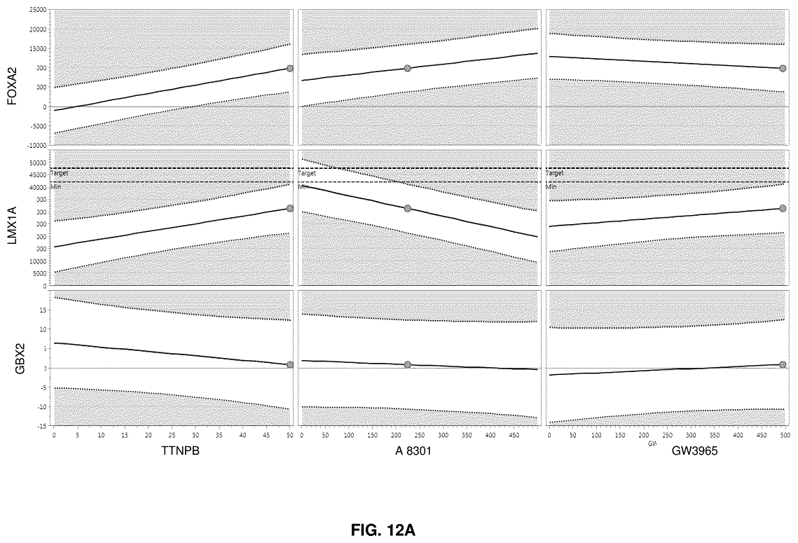

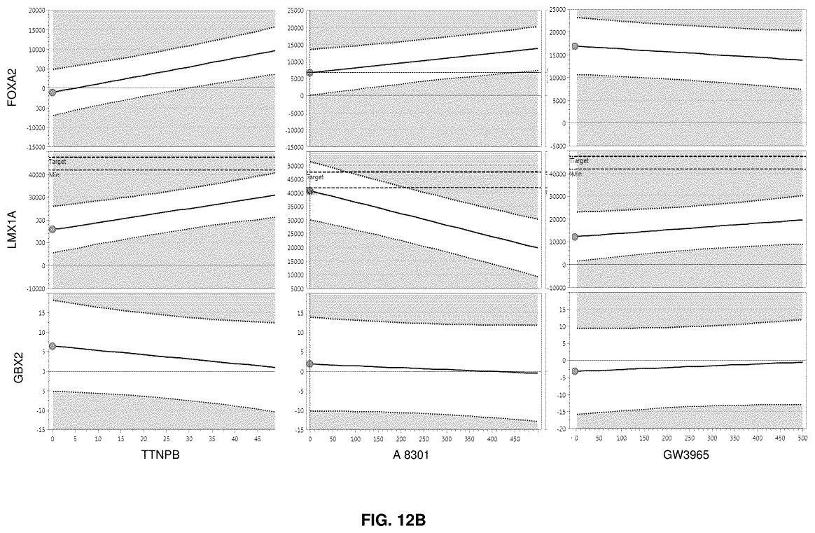

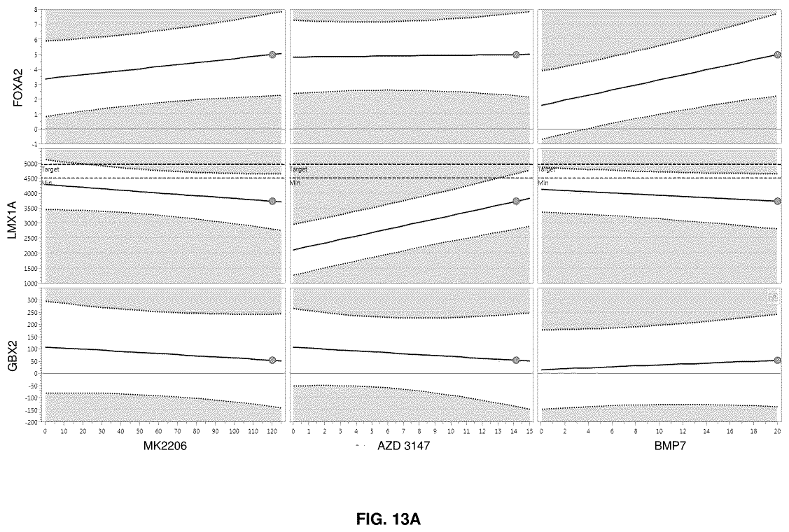

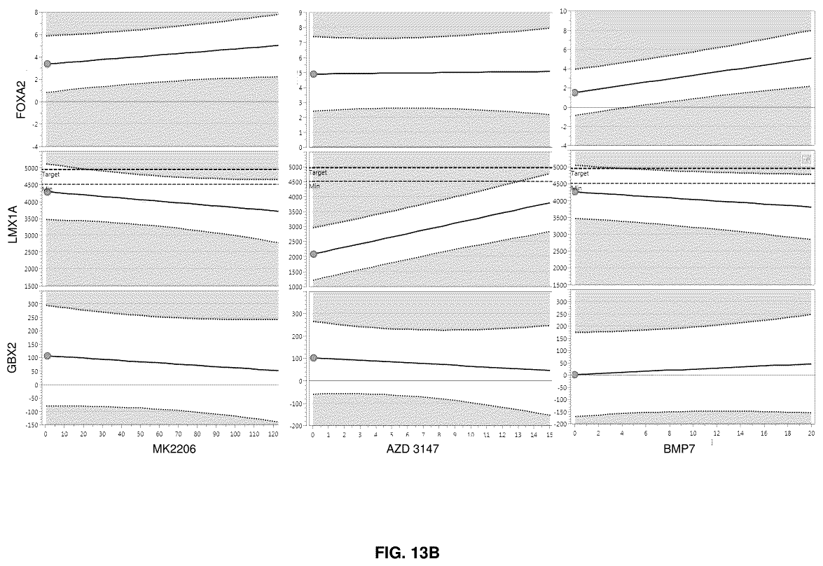

In the stage 2 recipe, each of the six finalized factors were removed while the other five factors were present and the expression levels of FOXA2, LMX1A and GBX2 were assessed, compared to the levels in the presence of all factors. According to the first experiment model, the absence of TTNPB lead to a rise of GBX2 expression, while the value of FOXA2 and LMX1A reduced drastically from 10,000 to 0 and 30,000 to 15,000, respectively. The absence of A 83-01 reduced the expression of FOXA2 from 10,000 to 7000, however, as expected, the value of LMX1A increased from 30,000 to 40,000. Deletion of GW3965 drastically reduced the value of LMX1A from 30,000 to 10,000 and increased the value of FOXA2 to 17,000 (FIG. 12A-B). According to the second experiment model, the absence of BMP7 and MK2206 both reduced the level of FOXA2 while increasing the value of LMX1A, with BMP7 as the main effector that led to 0 expression of FOXA2. The absence of AZD 3147 reduced the level of LMX1A from 4000 to 2000 while having almost no effect on value of FOXA2 (FIG. 13A-B) and therefore these factors were added to the final recipe.

### Example 3: Immunocytochemistry Validation of Stem Cell-Derived Midbrain Neural Progenitors Expressing FOXA2 and LMX1A

To further validate the culture protocol developed as described in Example 1, cells were treated with stage 1 and stage 2 differentiation media, and immunocytochemistry was used to assess expression of biomarkers of the midbrain region and neural progenitors at the end of each stage. Biomarkers tested included OTX2, a mesencephalic marker involved in positioning of midbrain and maintaining the mid-hindbrain boundary (Vernay et al. (2005) J. Neurosci. 25:4856-4867), LMX1A which is involved in generation and differentiation of midbrain dopaminergic progenitors (Yan et al. (2011) J. Neurosci. 31:12413-12425), FOXA2, which regulates generation of midbrain dopaminergic neurons at early and late stages of development (Ferri et al. (2007) Development 134:2761-2769), PAX2, which is expressed in midbrain and anterior hindbrain (Urbanek et al. (1997) Proc. Natl. Acad. Sci. USA 94:5703-5708), Nestin, which is an early neuronal marker, KI67, which is a proliferation marker and GBX2, which is a hindbrain marker.

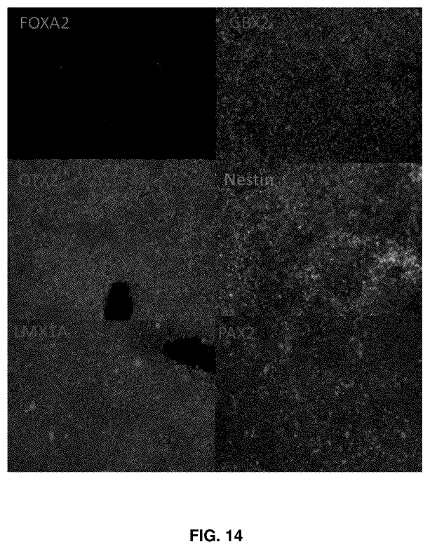

Immunocytochemistry images confirmed the expression of OTX2 and LMX1A in more than 90% of the cells by end of treatment with the stage 1 media. The markers PAX2, Nestin and KI67 were observed in some of the cells, as was some expression of GBX2. However, FOXA2 was not expressed (FIG. 14). After treatment with the stage 2 media, we observed that expression of LMX1A and OTX2 was maintained, while expression of FOXA2 was significantly increased, with detected of FOXA2 in more than 90% of the cells. GBX2 expression was also almost eliminated by end of stage 2 (FIG. 15). Detection of OTX2, LMX1A and FOXA2 by end of stage 2 of differentiation, while GBX2 was not detected, confirmed the recipes for stage 1 and stage 2 of differentiation of human induced pluripotent stem cells to midbrain neural progenitors after a 6-day treatment.

### Example 4: RNA-Seq Validation of Stem Cell-Derived Midbrain Neural Progenitors Expressing LMX1A and FOXA2

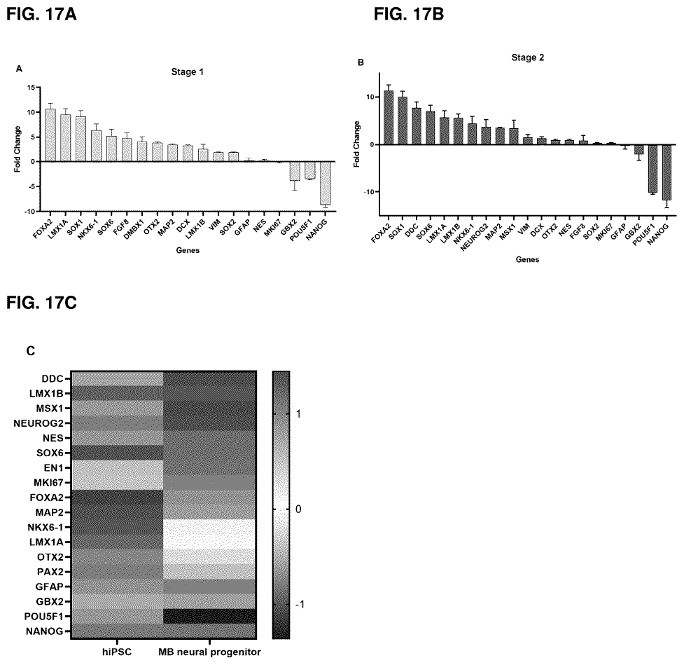

RNA sequencing was used to obtain the gene profile of cultured cells in the candidate recipes. Human iPSCs were cultured for a total of 6 days in stage 1 and 2 media and RNA of generated cells was sequenced at end of each stage. FIG. 17 shows normalized expression level of selected genes representative of midbrain region of the developing brain (OTX2, DMBX1, FOXA2, LMX1A), early neural identity (Nestin, SOX1, SOX2, Vimentin) and stem cell state (NANOG, POU5F1) in three replicates at day 0, day 3 and day 6. As it is shown in FIG. 17A and FIG. 17B, the level of stem cell genes has decreased in neural progenitors while the level of neuronal genes originating from midbrain region has increased; which validates the differentiation of hiPSCs to neural lineage with midbrain identity. FIG. 17A shows the fold change of 19 selected genes after 3 days treatment with stage 1 media compared to stage 0; FOXA2, LMX1A and SOX1 have the highest positive differential expression level (10.6, 9.5 and 9.1 respectively) compared to hiPSCs. Stemness genes NANOG and POU5F1, and also hindbrain gene GBX2, are at lowest levels (−8.7, −3.5 and −3.8 respectively). FIG. 17B shows the fold change of 21 selected genes of cells that were treated with stage 1 and stage 2 media compared to hiPSCs. FOXA2, SOX1 and DDC have the highest positive differential expression at 11.3, 10 and 7.7. Similar to stage 1, the lowest differential expression was observed with NANOG, POUF51 and GBX2. The heatmap of scaled gene profile of 18 selected genes of hiPSCs at day 0 and MB neural progenitors at day 6 (FIG. 17C) shows expression of midbrain progenitor genes, including DDC, LMX1A/B, SOX6, NEUROG2, FOXA2 and EN1, have increased after treatment with stage 1 and 2 media. The expression level of GFAP, a gene expressed in glial cells, was also observed and stayed the same during the 6-day differentiation, which shows the culture is mainly neuronal. This data demonstrates the ability of the stage 1 and stage 2 recipes as stage-wise differentiation media in directing the cells towards midbrain neuronal identity.

### Example 5: Culture Protocol Development for the Generation of Dopaminergic Neurons Expressing TH and KCNJ6

After treating the cells with stage 1 and stage 2 media to generate MB neural progenitors, as described in Examples 1-4, the MB neural progenitor cells are treated with stage 3 media for 3 days followed by stage 4 media for 14 days to differentiate them to dopaminergic neurons expressing TH and KCNJ6, based on the culture protocol developed in this example.

To develop the neuronal differentiation recipes, the impact of various agonist and antagonists (effectors) on maturation of MB neural progenitors was investigated using the HD-DoE method described in Example 1. These effectors were chosen based on available literature on developmental biology, differentiation of stem cells as well as mouse and human single cell RNA-seq data from midbrain neurons at the time.

As described further below, these experiments led to the generation of the stage 3 recipe shown below in Table 3 and the stage 4 recipe shown below in Table 4.

TABLE 3
Validated factors in stage 3 recipe

| Effectors | Role | concentration |
|---|---|---|
| BMP7 | BMP agonist | 15 ng/ml |
| AGN193109 | RAR antagonist | 100 nM |
| CHIR99021 | WNT agonist | 1 uM |
| PD0325901 | MEK antagonist | 100 nM |
| AZD3147 | mTOR antagonist | 15 nM |
| DBZ | Notch antagonist | 100 nM |

TABLE 4
Validated factors in stage 4 recipe

| Effectors | Role | concentration |
|---|---|---|
| BDNF | BDNF agonist | 10 ng/ml |
| GDNF | GDNF agonist | 10 ng/ml |
| Dopamine | Dopamine agonist | 10 uM |
| Heparin | Heparin or mimetic | 5 ug/ml |
| GW7647 | PPAR-a agonist | 250 nM |
| DBZ | Notch antagonist | 100 nM |

To engineer the recipe of stage 3 of differentiation, cells were first cultured in the stage 1 and stage 2 media as described herein, then they were treated with combinations of 8 or 12 factors for 3 days and the gene expression of cells in each condition was modeled. To guide the cells towards a subtype of midbrain dopaminergic neurons that reside in Substantia Nigra pars compacta (SNc) region of the brain, neural progenitors need to be SOX6⁺ (Pereira et al. (2021) Cell Rep. 37:109975 Oosterveen et al. (2021) Stem Cell Reports 16:2718-2735 Poulin et al. (2020) Trends Neurosci. 43:155-169). CORIN, NGN2 and MSX1 are other markers involved in neurogenesis of dopaminergic neurons originating from floorplate region of developing midbrain (Wang et al. (2020) Cells 9:1489; Samata et al. (2016) Nat Commun. 7:13097; Ono et al. (2007) Development 134:3213-3225; Prakash et al. (2006) J. Physiol. 575:403410).

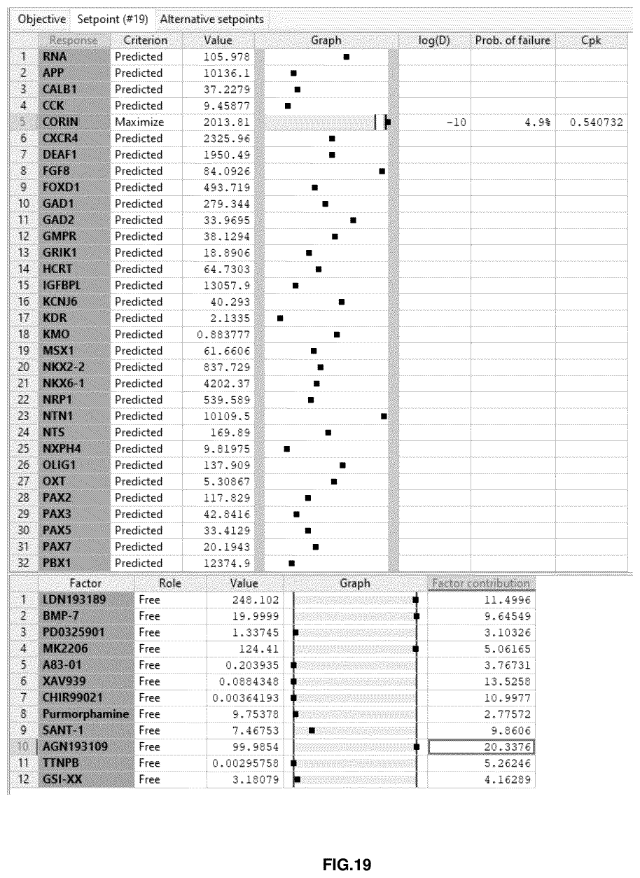

Therefore, we focused on maximizing expression of SOX6, CORIN, NGN2 and MSX1 in modeling experiments. One of the 12-factor models, the results of which are shown in FIG. 19, including LDN193189, AGN193109, BMP7, TTNPB, PD0325901, A8301, MHY1485, CHIR99021, XAV939, SANT-1, Purmorphamine and MK2206, provided a combination set that could maximize expression level of CORIN to more than 2000. In this model, AGN193109, a RA antagonist, had the most positive regulatory impact with a factor contribution of 20.3. Next two effectors with high positive factor contributions were LDN193189, an ALK1, ALK2, ALK3, ALK6 inhibitor and BMP7, respectively. XAV939, a WNT inhibitor, CHIR99021, a WNT activator, and SANT-1, a SHH antagonist, had the most negative impact on expression level of CORIN with factor contributions of 13.5, 10.9 and 9.9, respectively. Within the specifications of attaining 80% maximal expression of CORIN, this complex media composition had a Cpk value (process capability index) of 0.54, with a corresponding to a 4.9% risk of failure.

When the same model was optimized for maximum expression of MSX1 at 179, the factor with highest positive impact was BMP7 at 11.1 (FIG. 20). LDN193189, TTNPB, a RA agonist and XAV939 had the highest negative regulatory effect on its expression at 19.9, 15.5 and 11.2 respectively. Within the specifications of attaining 80% maximal expression of MSX1, this complex media composition had a Cpk value (process capability index) of 0.62, with a corresponding to a 3.1% risk of failure.

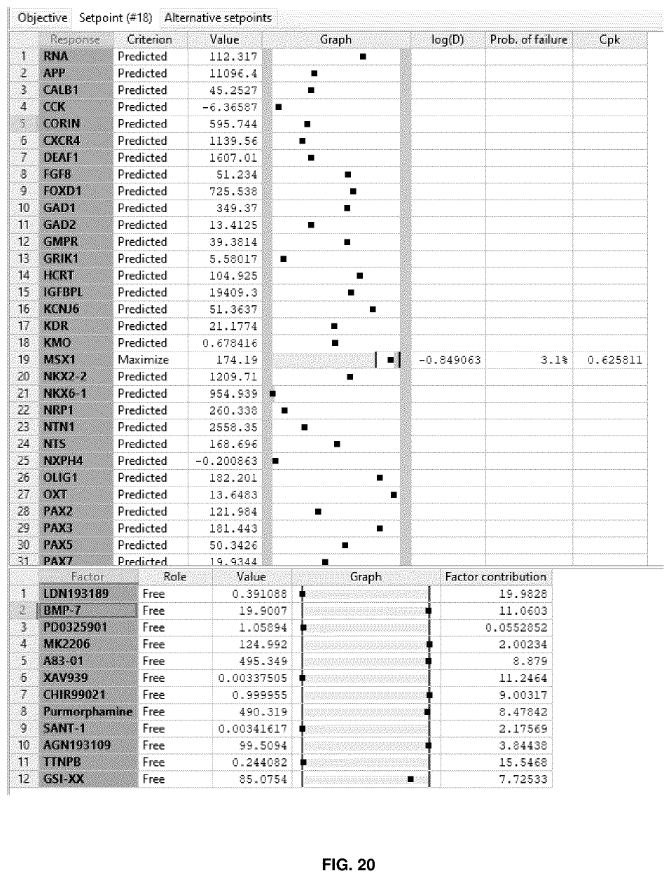

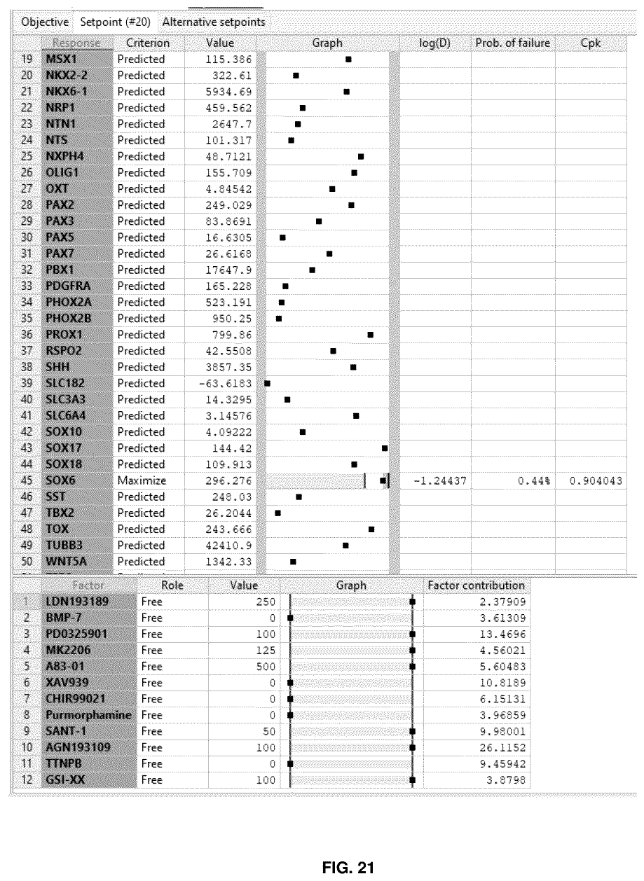

The model was also optimized for maximum expression of SOX6 at 296, which led to identifying two factors, AGN193109 and PD0325901 showing significant positive impacts on its expression with factor contributions of 26.1 and 13.5 (FIG. 21). XAV939 and TTNPB had the highest negative impact on optimization of this gene with factor contributions of 10.8 and 9.5, respectively. Within the specifications of attaining 80% maximal expression of SOX6, this complex media composition had a Cpk value (process capability index) of 0.9, with a corresponding to a 0.44% risk of failure.

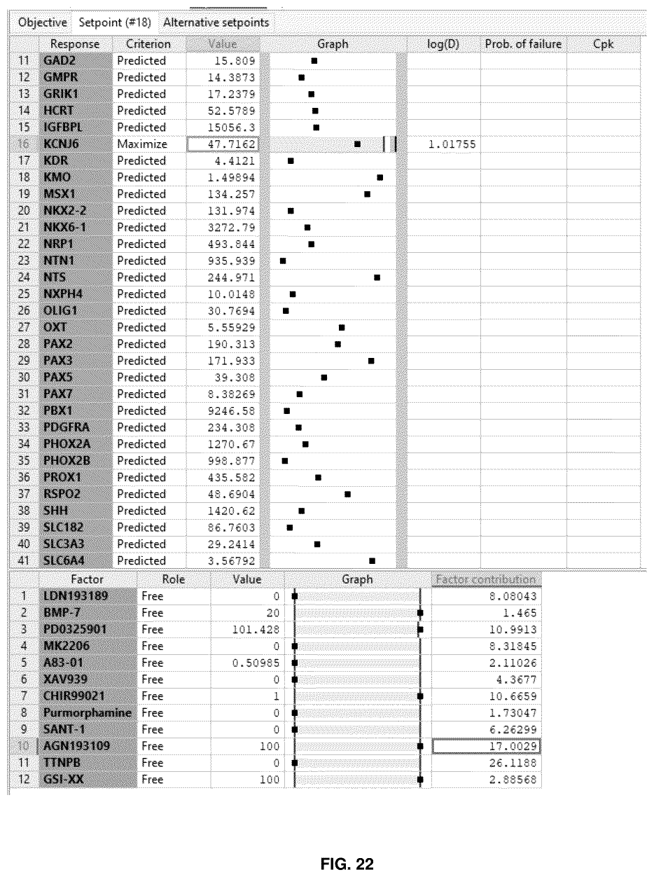

This 12-factor model resulted in elevated expression levels of KCNJ6 for the first time during differentiation experiments. KCNJ6, a G protein-activated potassium channel, is a terminal differentiation marker expressed by all human SNc and some VTA dopaminergic neurons (Reyes et al. (2012) J. Comp. Neural. 520:2591-607). Therefore, we also optimized the model for maximum expression of KCNJ6 at 47. Positive regulators of this gene were identified as AGN193109 at 17, PD0325901 at 10.9, CHIR99021 at 10.7 and BMP7 and DBZ with low factor contributions (FIG. 22).

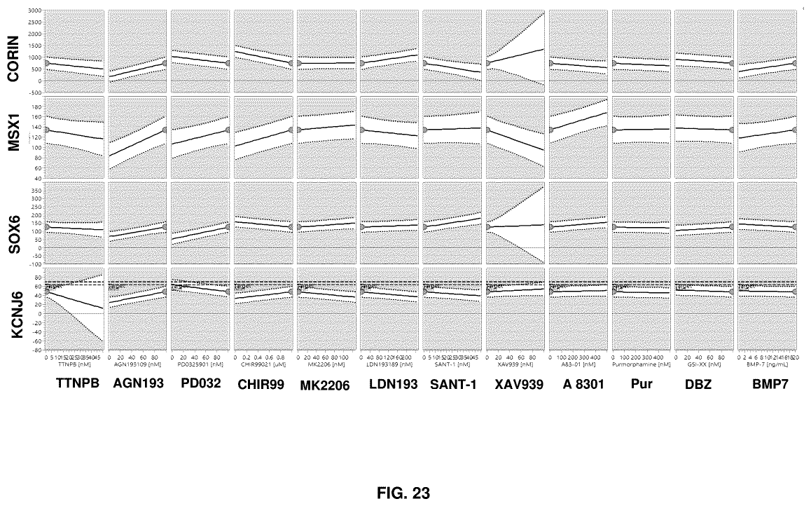

Next, we utilized dynamic profile analysis to find a combination set that can optimize expression of all four genes. Out of all effectors demonstrating high positive factor contributions on the selected genes, including AGN193109, PD0325901, BMP7, LDN193189 and CHIR99021, one factor LDN193189 had to be excluded due to its high negative impact on expression level of MSX1 (FIG. 23).

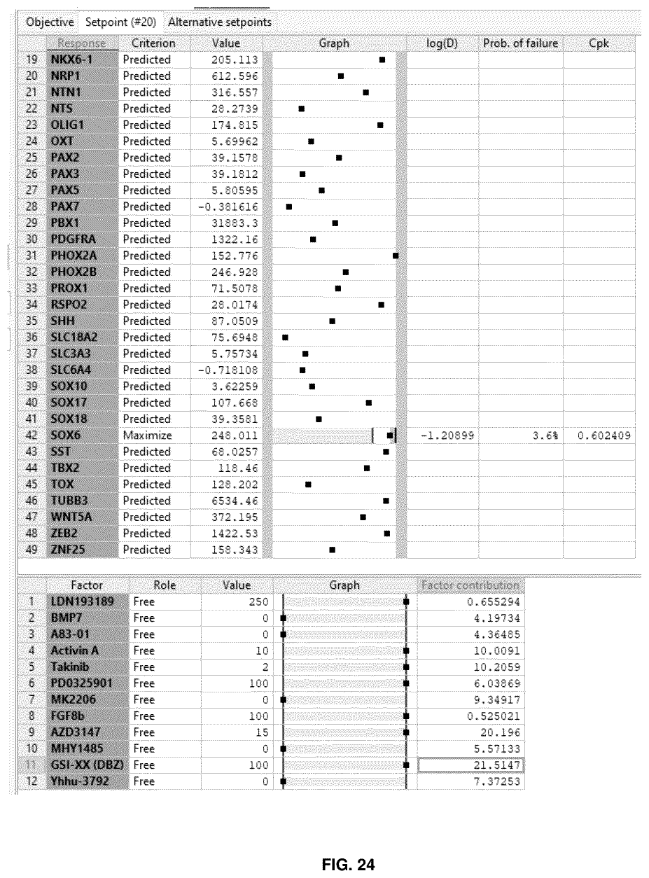

In another 12-factor model, additional factors including Activin A, Takinib (TAK inhibitor), FGF8b, AZD3147 (mTOR antagonist), MHY1485 (mTOR agonist), and Yhhu-3792 (Notch agonist) were tested. Some of the factors from previous model (BMP7, A 8301, LDN193189, PD0325901, MK2206 and DBZ) were also included. When optimized for SOX6 at 250, two new factors, DBZ and AZD3147 had the highest positive impact with factor contributions of 21.5 and 20.2 (FIG. 24). Within the specifications of attaining 80% maximal expression of SOX6, this complex media composition had a Cpk value (process capability index) of 0.60, with a corresponding to a 3.6% risk of failure.

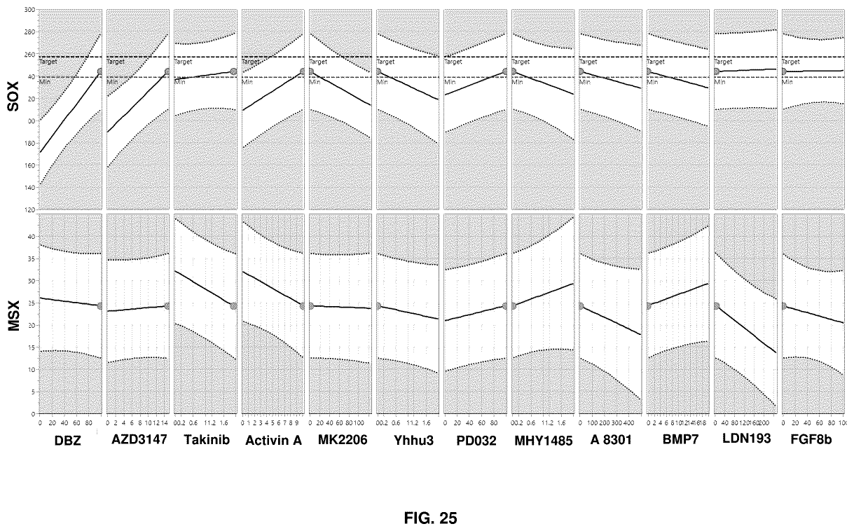

Since expression level of CORIN was significantly lower in this model, 20 compared to 2000, we did not optimize the model for its maximal expression. However, we analyzed the effect of these inputs on expression of MSX1 using dynamic profile analysis (FIG. 25). According to this model, Activin A and Takinib that had positive effect on SOX6, negatively regulate MSX1 and therefore they were excluded from the final recipe. AZD3147 and DBZ did not show any significant impact on expression level of MSX1.

We also observed positive regulatory effect of DBZ and AZD3147 on genes of interest in an 8-factor model, further confirming the analysis results of previous models (FIG. 26).

Therefore, considering these models, a candidate recipe for stage 3 consisting of BMP7, PD0325901, AGN193109, CHIR99021, AZD3147 and DBZ was made. This recipe was selected to maximize differentiation of cells as such related to robust and elevated expression of SOX6, KCNJ6, CORIN and MSX1. This recipe was further validated by immunocytochemistry assay (see Example 7).

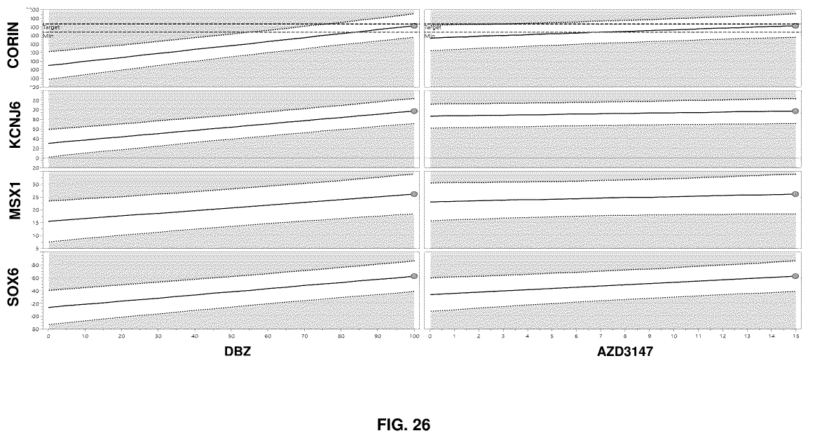

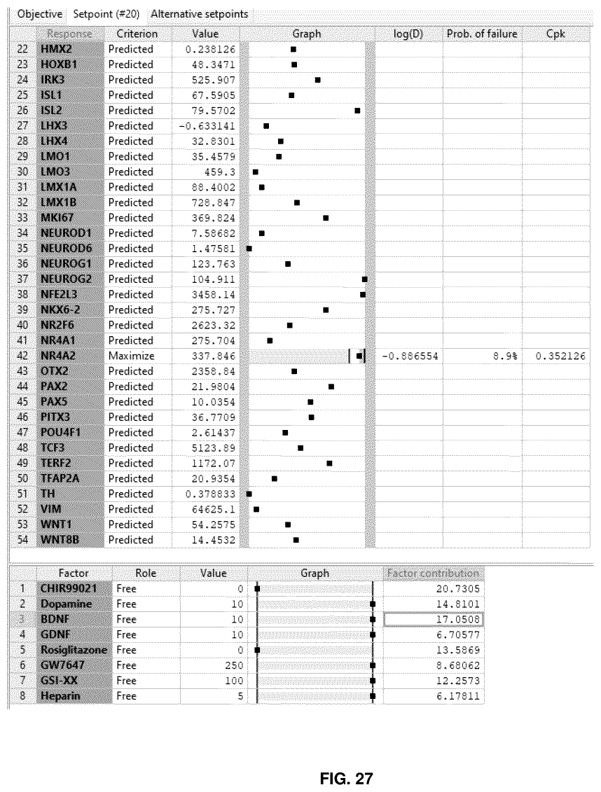

To further differentiate the cells to a mature neuronal identity, additional HD-DoE experiments were performed on the cells that were cultured in stage 1, stage 2 and stage 3 differentiation media. After 4 days, gene expression of cells in different combinatorial conditions were investigated. At this time, we focused on maximal expression of more mature dopaminergic genes such as NR4A2, PITX3 and TH (Tiklová et al. (2020) Nat Commun. 11:2434; Fiorenzano et al. (2021) Nat Commun., 12:7302). In one model, cells were treated with a combinatorial matrix with eight inputs including CHIR99021, BDNF, GDNF, Rosiglitazone, GW7647, DBZ and heparin. When maximized for NR4A2 at expression level 337.8, six factors demonstrated positive regulatory impact on its expression including BDNF with the highest factor contribution at 17, Dopamine, DBZ, GW7647, GDNF and heparin (FIG. 27). While CHIR99021 and Rosiglitazone had significant negative impact on NR4A2 with factor contributions of 20.7 and 13.6, respectively. Within the specifications of attaining 80% maximal expression of NR4A2, this complex media composition had a Cpk value (process capability index) of 0.35, with a corresponding to 8.9% risk of failure.

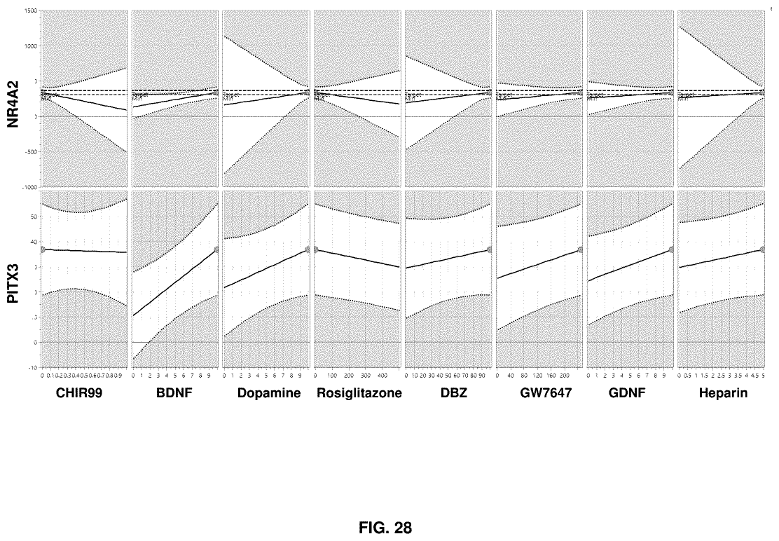

The impact of the compounds on expression level of PITX3 was investigated by using dynamic profile analysis and similar trends were observed for both genes except for CHIR99021 that did not have a meaningful effect on expression level of PITX3 (FIG. 28).

Considering this model, conditions that maximize differentiation of cells to SNc region with dopaminergic neuronal cell identity as such relate to robust and elevated expression of NR4A2 and PITX3 included the following effector inputs: BDNF, GDNF, Dopamine, GW7647, DBZ and heparin. This stage 4 recipe was further validated by immunocytochemistry assay (see Example 7).

### Example 6: Factor Criticality Analysis of Dopaminergic Neuron-Inducing Culture Conditions

To assess the impact of elimination of each validated factor identified in Example 5 for the stage 3 and stage 4 recipes, we used dynamic profile analysis and compared the expression level of genes of interest in absence of each finalized factor while others are present. Since expression level of genes of interest reveal whether the desired outcome is reachable, this factor criticality analysis revealed the extent of importance of each input effector.

In the stage 3 recipe, each of the finalized factors were removed in their respective models while other factors were present and the expression level of genes of interest was assessed compared to presence of all factors. Results are shown in FIG. 29A and FIG. 29B. When AGN193109 was excluded, level of all CORIN, KCNJ6, MSX1 and SOX6 decreased from 900 to 450, 50 to 35, 140 to 110 and 105 to 60, respectively. When BMP7 was removed expression levels of CORIN and MSX1 were decreased to 500 and 100, however, level of KCNJ6 and SOX6 did not have a significant change. When PD0325901 was removed, expression levels of CORIN and KCNJ6 increased to 1200 and 70 while levels of MSX1 and SOX6 decreased to 105 and 20. In the absence of CHIR99021, the expression level of CORIN was again increased to 1200 while the level of KCNJ6 and MSX1 dropped to 50 and 115. The level of SOX6 was also increased to 125. Out of the selected genes, the expression level of CORIN is the highest, and even with the negative regulatory impact coming from PD0325901, it's still at a significant high level (900), while SOX6 drops significantly from 105 to 20 if PD0325901 is removed. Therefore, it was decided to include this compound in the final recipe. CHIR99021 had a similar effect on CORIN, but its absence impacted MSX1 significantly in a negative way. Therefore, CHIR99021 was also included in the final recipe.

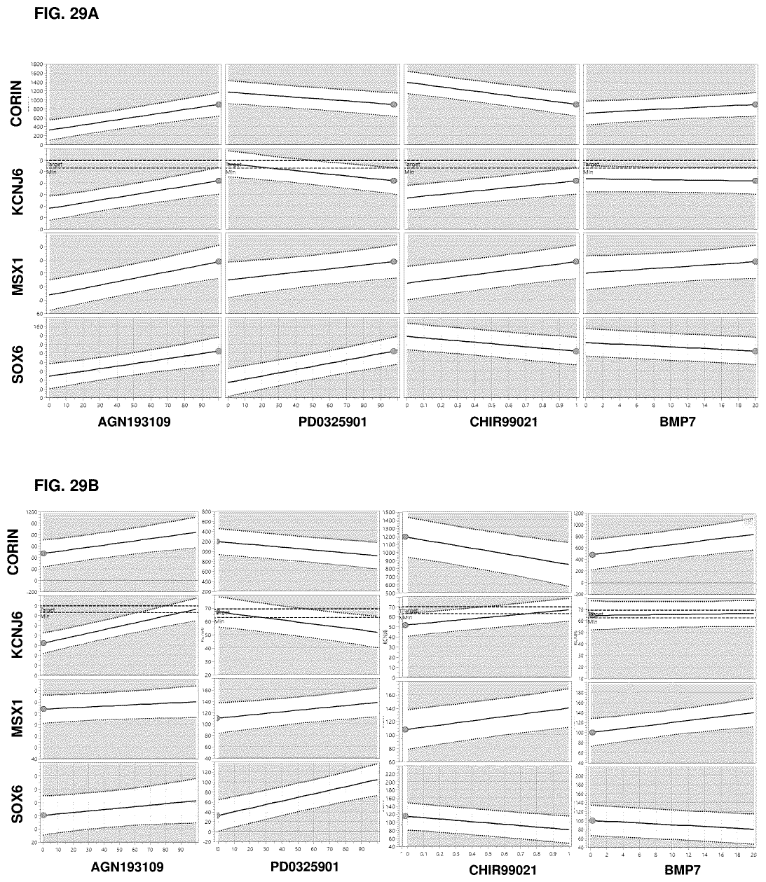

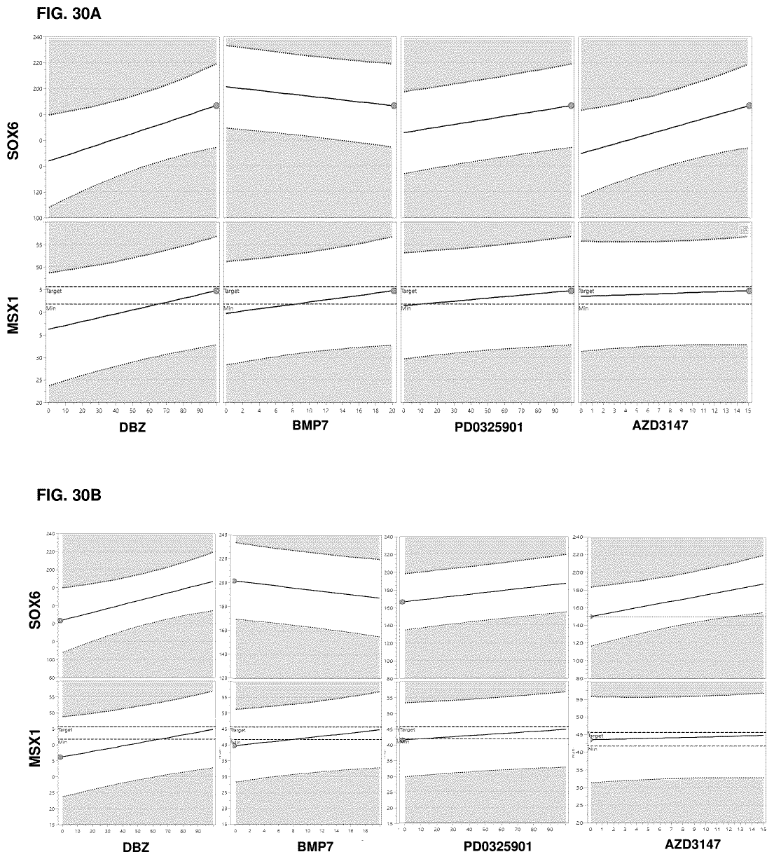

In another model, to determine the combinatorial effect of AZD3147 and DBZ on gene profile of cells in presence of other factors of final recipe, the expression levels of SOX6 and MSX1 were compared in the absence of each of the factors in the presence of PD0325901 and BMP7. These two compounds were the only final compounds that were included in inputs of this experimental model The results are shown in FIG. 30A and FIG. 30B. When AZD3147 was excluded from the combination set, the level of SOX6 dropped from 185 to 150 while MSX1 did not change. When DBZ was removed, the level of SOX6 and MSX1 both decreased to 140 and 35, respectively.

As a validatory analysis, we also analyzed expression level of these two genes when PD0325901 was absent, and it was confirmed that it leads to decrease in expression of SOX6 from 185 to 165 and MSX1 from 45 to 40.

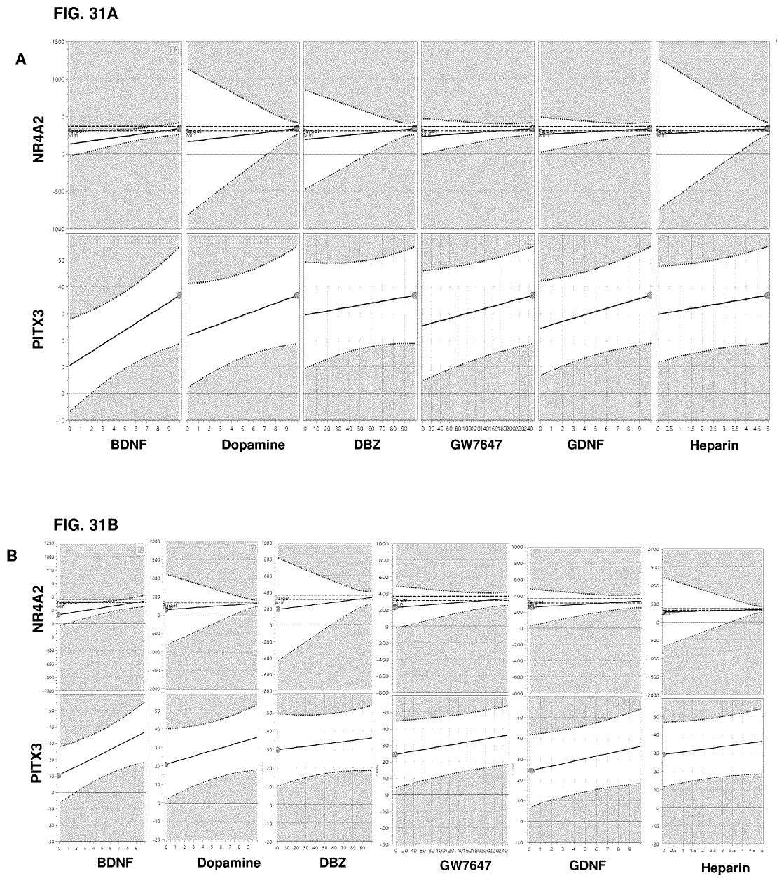

In the stage 4 recipe, each of six finalized factors were excluded while other factors were present and the expression levels of NR4A2 and PITX3 were assessed compared to the presence of all factors. The results are shown in FIG. 31A and FIG. 31B. In the absence of BDNF, expression of both PITX3 and NR4A2 decreased from 40 to 10 and 400 to 100. When Dopamine was excluded from the recipe, the expression level of NR4A2 almost reached 0 and PITX3 dropped to 20. When GDNF was removed, the expression level of NR4A2 decreased to 300 and PITX3 to 25. When DBZ was removed, the level of NR4A2 decreased to 200 again and PITX3 reached 30. When GW7647 was excluded, a similar trend was observed which resulted in lower expression levels for both genes. Only in the absence of heparin, the expression level of NR4A2 did not significantly change, but still it caused a drop in expression of PITX3 to 30.

Factor criticality analysis demonstrated the importance of inclusion of each of the compounds in recipes of stage 3 and stage 4 differentiation media.

### Example 7: Immunocytochemistry Validation of Midbrain Dopaminergic Neurons Expressing TH and KCNJ6

To further validate the developed recipes of Example 5, cells were first treated with the stage 1 and stage 2 differentiation media according to Example 1 and then treated with the stage 3 differentiation media for 3 days each and stage 4 media for 14 days, then standard immunocytochemistry assay was used to assess expression of biomarkers of immature neurons of ventral midbrain region at the end of stage 3 and mature neurons at the end of stage 4. Biomarkers included midbrain neuronal specific markers such as SOX6, ALDH1A1, MSX1, TH, NURR1 (NR4A2), PITX3 and KCNJ6 along with mature pan neuronal markers such as TUBB3, MAP2, Neurofilament (NF) and SYN1.

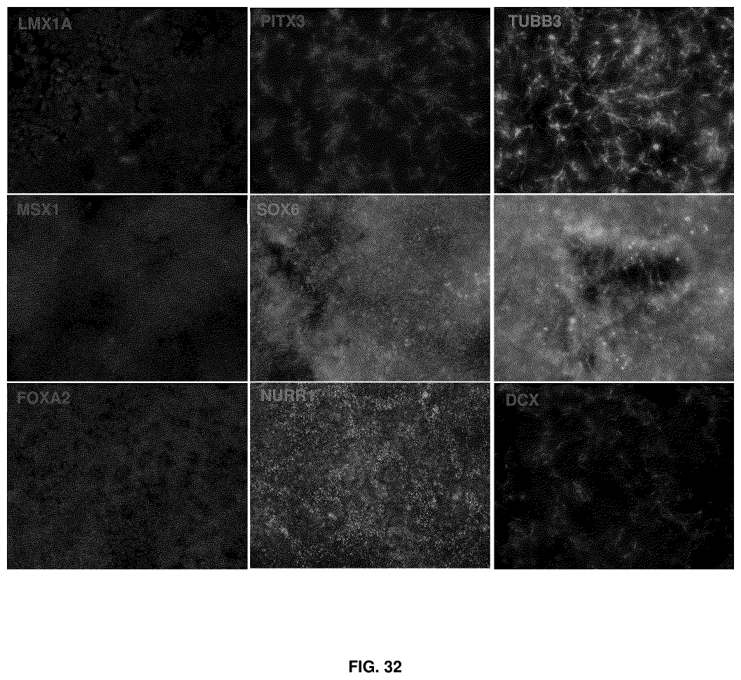

Immunocytochemistry images confirmed the expression of SOX6 and MSX1 by end of stage 3 in more than 90% of the cells (FIG. 32). We also detected neuronal markers beta-tubulin III (TUBB3), immature neuronal marker Doublecortin (DCX) and initial expression of post mitotic dopaminergic neurons PITX3 and NURR1, as early as day 9 in vitro.

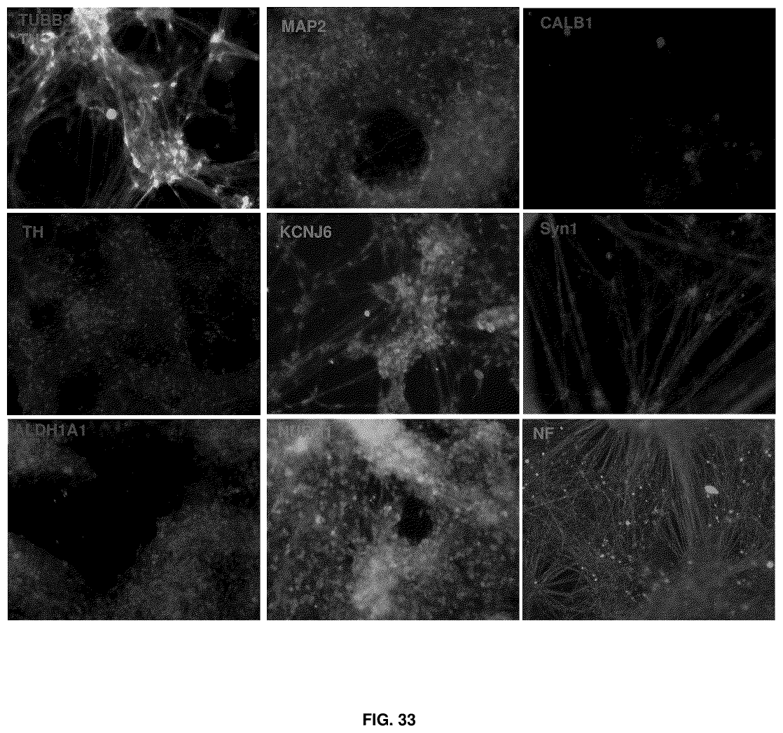

Images of mature neurons at the end of stage 4, confirmed cells in culture express mature dopaminergic markers such as KCNJ6 and TH. We also observed most cells in culture express mature pan neuronal markers including MAP2, NF and Syn1 (FIG. 33). We stained the cells for competing fate, dopaminergic neurons of ventral tegmental area (VTA) that express CALB1 (Brignani et al. (2017) Front. Nefiroanoi. 11:55) and detected that less than 10% of cells express it. At this point expression of NURR1 and ALDH1A1 was observed in most cells.

Detection of TH and KCNJ6 by the end of stage 4 of differentiation, while CALB1 was not detected, confirmed the robustness and high conversion potential of of the stage-wise recipes described herein for differentiation of human induced pluripotent stem cells to SNc dopaminergic neurons after 23 days in vitro.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A method of generating human FOXA2+ LMX1A+ MSX1+ PITX3+ DCX+ immature midbrain neurons comprising:
culturing human OTX2+ FOXA2+ LMX1A+ midbrain neural progenitor cells (MB NPCs) in a culture media comprising a WNT pathway agonist, an mTOR pathway antagonist, a retinoic acid receptor (RAR) pathway antagonist, a MEK pathway antagonist, a Notch pathway antagonist and a BMP pathway agonist for three days (day 0-day 3) to obtain human FOXA2+ LMX1A+ MSX1+ PITX3+ DCX+ immature midbrain neurons (MB-immature neurons).

2. The method of claim 1, further comprising culturing the MB-immature neurons in a culture media comprising a BDNF pathway agonist, a GDNF pathway agonist, a PPAR-a pathway agonist, heparin or heparin mimetic, a Notch pathway antagonist and a dopamine agonist for at least fourteen days to obtain TH+ KCNJ6+ mature dopaminergic neurons.

3. A method of generating human TH+ KCNJ6+ mature dopaminergic neurons comprising:
(a) culturing human OTX2+ FOXA2+ LMX1A+ midbrain neural progenitor cells (MB NPCs) in a culture media comprising a WNT pathway agonist, an mTOR pathway antagonist, a retinoic acid receptor (RAR) pathway antagonist, a MEK pathway antagonist, a Notch pathway antagonist and a BMP pathway agonist for three days (day 0-day 3) to obtain human FOXA2+ LMX1A+ MSX1+ PITX3+ DCX+ immature midbrain neurons (MB-immature neurons); and
(b) culturing the MB-immature neurons in a culture media comprising a BDNF pathway agonist, a GDNF pathway agonist, a PPAR-a pathway agonist, heparin or heparin mimetic, a Notch pathway antagonist and a dopamine agonist for at least fourteen days (day 3-day 17) to obtain TH+ KCNJ6+ mature dopaminergic neurons.

4. A method of generating human TH+ KCNJ6+ mature dopaminergic neurons comprising:
(a) culturing human pluripotent stem cells in a culture media comprising a WNT pathway agonist, an SHH pathway agonist, a BMP pathway antagonist, an AKT pathway antagonist and a MEK pathway antagonist on days 0-3 to obtain committed midbrain neural stem cells (MB NSCs);
(b) culturing the MB NSCs in a culture media comprising a BMP pathway agonist, an RA pathway agonist, an LXR pathway agonist, an AKT pathway antagonist, an mTOR pathway antagonist and a TGF pathway antagonist on days 4-6 to obtain human OTX2+ FOXA2+ LMX1A+ midbrain neural progenitor cells (MB NPCs);
(c) culturing the MB NPCs in a culture media comprising a WNT pathway agonist, an mTOR pathway antagonist, a retinoic acid receptor (RAR) pathway antagonist, a MEK pathway antagonist, a Notch pathway antagonist and a BMP pathway agonist on days 6-9 to obtain human FOXA2+ LMX1A+ MSX1+ PITX3+ DCX+ immature midbrain neurons (MB-immature neurons); and
(d) culturing the MB-immature neurons in a culture media comprising a BDNF pathway agonist, a GDNF pathway agonist, a PPAR-a pathway agonist, heparin or heparin mimetic, a Notch pathway antagonist and a dopamine agonist on days 9-23 to obtain TH+ KCNJ6+ mature dopaminergic neurons.

5. The method of claim 4, wherein the human pluripotent stem cells are induced pluripotent stem cells (iPSCs).

6. The method of claim 4, wherein the human pluripotent stem cells are embryonic stem cells.

7. The method of claim 1, wherein the WNT pathway agonist is selected from the group consisting of CHIR99021, CHIR98014, SB 216763, SB 415286, LY2090314, 3F8, A 1070722, AR-A 014418, BIO, BIO-acetoxime, AZD1080, WNT3A, alsterpaullone, indirubin-3-oxime, 1-azakenpaullone, kenpaullone, TC-G 24, TDZD 8, TWS 119, NP 031112, AT 7519, KY 19382, AZD2858, and combinations thereof.

8. The method of claim 7, wherein the WNT pathway agonist is present in the culture media at a concentration within a range of 0.5-2.0 μM.

9. The method of claim 7, wherein the WNT pathway agonist is CHIR99021, which is present in the culture media at a concentration of 1.0-1.1 μM.

10. The method of claim 1, wherein the mTOR pathway antagonist is selected from the group consisting of AZD3147, rapamycin, sirolimus, temsirolimus, everolimus, ridaforolimus, umirolimus, zotarolimus, torin-1, torin-2, vistusertib, MHY1485, AZD8055, dactolisib, PI-103, NU7441, BC-LI-0186, eCF 309, ETP 45658, niclosamide, omipalisib, PF 04691502, PF 05212384, WYE 687, XL 388, STK16-IN-1, PP 242, torkinib, sapanisertib, voxtalisib, and combinations thereof.

11. The method of claim 10, wherein the mTOR pathway antagonist is present in the culture media at a concentration within a range of 10-30 nM.

12. The method of claim 10, wherein the mTOR pathway antagonist is AZD3147, which is present in the culture media at a concentration of 15 nM.

13. The method of claim 1, wherein the RAR pathway antagonist is selected from the group consisting of AGN193109, BMS 195614, CD 2665, ER 50891, LE 135, LY 2955303, MM11253, and combinations thereof.

14. The method of claim 13, wherein the RAR pathway antagonist is present in the culture media at a concentration within a range of 50-250 nM.

15. The method of claim 13, wherein the RAR pathway antagonist is AGN193109, which is present in the culture media at a concentration of 100 nM.

16. The method of claim 1, wherein the MEK pathway antagonist is selected from the group consisting of PD0325901, Binimetinib (MEK162), Cobimetinib (XL518), Selumetinib, Trametinib (GSK1120212), CI-1040 (PD-184352), Refametinib, ARRY-142886 (AZD-6244), PD98059, U0126, BI-847325, RO 5126766, BIX 02189, Pimasertib, TAK 733, AZD8330, PD318088, SL 327, GDC 0623, R05126766, Myricetin, and combinations thereof.

17. The method of claim 16, wherein the MEK pathway antagonist is present in the culture media at a concentration within a range of 50-250 nM.

18. The method of claim 16, wherein the MEK pathway antagonist is PD0325901, which is present in the culture media at a concentration of 100-110 nM.

19. The method of claim 1, wherein the Notch pathway antagonist is selected from the group consisting of DBZ, avagacestat, begacestat, BMS 299897, Compound E, DAPT, JLK6, L-685,458, LY 450139, MRK 560, PF 3084014 hydrobromide, LY 3039478, LY 411575, RO 4929097, and combinations thereof.

20. The method of claim 19, wherein the Notch pathway antagonist is present in the culture media at a concentration within a range of 50-250 nM.

21. The method of claim 19, wherein the Notch pathway antagonist is DBZ, which is present in the culture media at a concentration of 100 nM.

22. The method of claim 1, wherein the BMP pathway agonist is selected from the group consisting of BMPs, sb4, ventromorphins, and combinations thereof.

23. The method of claim 22, wherein the BMP pathway agonist is present in the culture media at a concentration within a range of 5-50 ng/ml.

24. The method of claim 22, wherein the BMP pathway agonist is BMP7, which is present in the culture media at a concentration of 10-15 ng/ml.

25. The method of claim 2, wherein the BDNF pathway agonist is selected from the group consisting of BDNF, rotigotine, 7,8-DHF, ketamine, tricyclic dimeric peptide-6 (TDP6), LM22A-4, and combinations thereof.

26. The method of claim 25, wherein the BDNF pathway agonist is present in the culture media at a concentration within a range of 5-50 ng/ml.

27. The method of claim 25, wherein the BDNF pathway agonist is BDNF, which is present in the culture media at a concentration of 10 ng/ml.

28. The method of claim 2, wherein the GDNF pathway agonist is selected from the group consisting of GDNF, BT13, BT44, and combinations thereof.

29. The method of claim 28, wherein the GDNF pathway agonist is present in the culture media at a concentration within a range of 5-50 ng/ml.

30. The method of claim 28, wherein the GDNF pathway agonist is GDNF, which is present in the culture media at a concentration of 10 ng/ml.

31. The method of claim 2, wherein the PPAR-a pathway agonist is selected from the group consisting of GW7647, fenofibrate, fenofibrate-d6, WY 14643, CP 775146, CP 868388 free base, Tesaglitazar, Oleylethanolamide, Oleyolethanolamide-d2, Oleyolethanolamide-d4, PPAR agonist 1, Clofibrate, Clofibrate-d4, Wistin, Indeglitazar, Netoglitazone, GW0742, Bezafibrate, Bezafibrate-d4, Chiglitazar, BMS 687453, Lanifibranor, Saroglitazar, Saroglitazar Magnesium, Saroglitazar-d5, Imiglitazar, AVE-8143, GW 590735, Ertiprotafib, LJ 570, Seladelpar Sodium Salt, Edaglitazone, Muraglitazar, Ragaglitazar, GW 9578, MHY 908, KRP-297, Elafibranor, Aleglitazar, AM3102, Eupatilin, Clofibric acid, Clofibric acid-d4, Naveglitazar, Naveglitazar racemate, and combinations thereof.

32. The method of claim 31, wherein the PPAR-a pathway gonist is present in the culture media at a concentration within a range of 200-300 nM.

33. The method of claim 31, wherein the PPAR-a pathway agonist is GW7647, which is present in the culture media at a concentration of 250 nM.

34. The method of claim 2, wherein heparin or heparin mimetic is selected from the group consisting of heparin, heparan sulfate, enoxaparin, small molecular weight heparins, AV5026, M402, and combinations thereof.

35. The method of claim 34, wherein heparin or heparin mimetic is present in the culture media at a concentration within a range of 2-8 μg/ml.

36. The method of claim 34, wherein the culture media comprises heparin, which is present in the culture media at a concentration of 5 μg/ml.

37. The method of claim 2, wherein the dopamine agonist is selected from the group consisting of dopamine, dopamine hydrochloride, (R)-(−)-Apomorphine hydrochloride, Bromocriptine mesylate, Bromocriptine-13C,d3, CNV dopamine, Dehydroergotamine mesylate, Lisuride, Lisuride maleate, Mesulergine hydrochloride, Piribedil dihydrochloride, Piribedil, Quinelorane hydrochloride, (−)-Quinpirole hydrochloride, Ropinirole, Tau-aggregation-IN-1, NMI 8739, U91356, Foscarbidopa, Quinagolide hydrochloride, Dexpramipexole dihydrochloride, PD-168077 maleate, Rotigotine hydrochloride, PF592379, Rotigotine, (Rac)-Rotigotine hydrochloride, (Rac)-Rotigotine-d7 hydrochloride, Ro 10-5824 dihydrochloride, (+)-Dihydrexidine hydrochloride, Talipexole, PF2562, Neuromedin N, A68930, A68930 hydrochloride, A77636 dihydrochloride, PD 119819, PD 128907 hydrochloride, CY 208-243, Dexpramipexole, Dexpramipexole-d3 dihydrochloride, Dexpramipexole-d7 dihydrochloride, SKF 38393 hydrochloride, SKF 38393 hydrobromide, SKF 82958, ABT 670, ABT-724, ABT-724 trihydrochloride, (R)-PF-06256142, Talipexole dihydrochloride, R-Preclamol, Pardoprunox hydrochloride, Pardoprunox, Pergolide mesylate, BP897, BP897 hydrochloride, LY 3154207, Tavapadon, Roxindole, UNC994, Cabergoline, Brexpiprazole, Pergolide-d7 mesylate, Cabergoline-d6, Roxindole hydrochloride, WAY-100635 Maleate, Sibenadet hydrochloride, Dihydrexidine, Dihydrexidine hydrochloride, Bifeprunox, OS-3-106, Brilaroxazine, Pramipexole dihydrochloride hydrate, Pramipexole, Pramipexole dihydrochloride, Cabergoline-d5, Perospirone, Brexpiprazole-d8, (Rac)-Tavapadon, ML417, Brexpiprazole S-oxide, Pramipexole-d7 dihydrochloride, Pramipexole-d5 dihydrochloride, UCSF924, WAY-100635, SKF83959, Pramipexole (N-Propyl-3,3,3-d3) (dihydrochloride), Sarizotan, Brexpiprazole S-oxide D8, SKF 83959 hydrobromide, and combinations thereof.

38. The method of claim 37, wherein the dopamine agonist is present in the culture media at a concentration within a range of 5-15 μM.

39. The method of claim 37, wherein the dopamine agonist is dopamine, which is present in the culture media at a concentration of 10 μM.

40. A culture media for obtaining human midbrain immature neurons according to the method of claim 1, the media comprising a WNT pathway agonist, an mTOR pathway antagonist, a retinoic acid receptor (RAR) pathway antagonist, a MEK pathway antagonist, a Notch pathway antagonist and a BMP pathway agonist.

41. A culture media for obtaining human mature dopaminergic neurons according to the method of claim 2, comprising a BDNF pathway agonist, a GDNF pathway agonist, a PPAR-a pathway agonist, heparin or heparin mimetic, a Notch pathway antagonist and a dopamine agonist.

42. An isolated cell culture of human midbrain immature neurons obtained by the method of claim 1, the culture comprising: human FOXA2+ LMX1A+ MSX1+ PITX3+ DCX+ immature midbrain neurons cultured in a culture media comprising a WNT pathway agonist, an mTOR pathway antagonist, a retinoic acid receptor (RAR) pathway antagonist, a MEK pathway antagonist, a Notch pathway antagonist and a BMP pathway agonist.

43. An isolated cell culture of human mature dopaminergic neurons obtained by the method of claim 2, the culture comprising: human TH+ KCNJ6+ mature dopaminergic neurons cultured in a culture media comprising a BDNF pathway agonist, a GDNF pathway agonist, a PPAR-a pathway agonist, heparin or heparin mimetic, a Notch pathway antagonist and a dopamine agonist.

44. Human FOXA2+ LMX1A+ MSX1+ PITX3+ DCX+ immature midbrain neurons generated by the method of claim 1.

45. Human TH+ KCNJ6+ mature dopaminergic neurons generated by the method of claim 2.
